SEQUENCE LISTING (183 sequences)

SEQ ID NO: 1 - protein, 5 aa, Arabidopsis thaliana
GDDDD

SEQ ID NO: 2 - protein, 6 aa, Arabidopsis thaliana
  MISC_FEATURE (2)..(2): X can be any naturally occuring amino acid
DXAPAA

SEQ ID NO: 3 - protein, 14 aa, Arabidopsis thaliana
GDDDDSGYDYAPAA

SEQ ID NO: 4 - protein, 14 aa, Arabidopsis thaliana
GDDDDDDCDVAPAA

SEQ ID NO: 5 - protein, 17 aa, Glycine max
GDDDDDDNGVIDVAPAA

SEQ ID NO: 6 - protein, 15 aa, Glycine max
GDDDDDGGYDYAPAA

SEQ ID NO: 7 - protein, 16 aa, Glycine max
GDDDDDDGGYDYAPAA

SEQ ID NO: 8 - protein, 14 aa, Oryza sativa
GDDDDDDYDCAPAA

SEQ ID NO: 9 - protein, 14 aa, Oryza sativa
GDDDDDDVDVAPAA

SEQ ID NO: 10 - protein, 39 aa, Artificial Sequence
  consensus seuqence shown in Fig. 2D
MXVFSKNXDGVXWAATVSXAENEGDDDDDDGYDVAPAAX

SEQ ID NO: 11 - protein, 56 aa, Aegilops tauschii
MAPASKVMSHIVQDGGIADYAVYAAAPCDAWCGGRHRKAESDGDDDDDDYDCAPAA

SEQ ID NO: 12 - protein, 36 aa, Amborella trichopoda
MLQRYDAPFVGQKWHQKRIGEGDDDDDDDYDCAPAA

SEQ ID NO: 13 - protein, 36 aa, Amborella trichopoda
MLQRYDAPFVGQKWHQKRIGEGDDDDDDDYDFAPAA

SEQ ID NO: 14 - protein, 39 aa, Amborella trichopoda
MLDRHDAHLGCQKWHQKKILRTEGDDDDDDDDYDCAPAT

SEQ ID NO: 15 - protein, 39 aa, Amborella trichopoda
MLDRHDAHLCCLKWHQKKILRTEGDDDDDDDDYDCAPAA

SEQ ID NO: 16 - protein, 26 aa, Amborella trichopoda
MASEENPPNRRDDDDDDDDYDCAPAT

SEQ ID NO: 17 - protein, 23 aa, Amborella trichopoda
MASEENREGDDDDDDDYDCAPAA

SEQ ID NO: 18 - protein, 50 aa, Arabidopsis lyrata
MMSFVANLAIKSLDRASAVYVEDVVDSSRVAYGENGGDDDDSGYDYAPAA

SEQ ID NO: 19 - protein, 50 aa, Arabidopsis lyrata
MMSFVANLVIKSFYRASAMYVEDMVDSSRATCLENGGDDDDSGYDYAPAA

SEQ ID NO: 20 - protein, 50 aa, Arabidopsis lyrata
MMYFFANLVSKSFDRASAVYVEDVVDCSRATCVENGGDDDDSGYDYAPAA

SEQ ID NO: 21 - protein, 56 aa, Arabidopsis lyrata
MISVTEFILCIDDNVGGTCIGGEVVISGQAFVYAQSVYVEDGDNDDDDIYDYAPAA

SEQ ID NO: 22 - protein, 56 aa, Arabidopsis lyrata
MISVSEIVLYIHENVYETSIGVNIANNDKVFEYAQATFVENGDNDDDVIYDYAPAA

SEQ ID NO: 23 - protein, 56 aa, Arabidopsis lyrata
MSSLSEFVLSIYDHVSESCVGSDTTSYDQEIKSRQAAYAENGDQDDDDIYDYAPAA

SEQ ID NO: 24 - protein, 56 aa, Arabidopsis lyrata
MVSIYKFVLCKCDQVRETCIRGDVTYNNGEFEYHQVAFIENGDDDDDIIYDYAPAA

SEQ ID NO: 25 - protein, 50 aa, Arabidopsis thaliana
MMSFVANLAIKRFDHASTVYVEDVVDSSRVAYSENGGDDDDSGYDYAPAA

SEQ ID NO: 26 - protein, 50 aa, Arabidopsis thaliana
MMSYVANLVIKSFDRASVVYVEDVVDSSRATCVENGGDDDDSGYDYAPAA

SEQ ID NO: 27 - protein, 47 aa, Arabidopsis thaliana
MAVVSHNNAEGRLYESTQTWPIAYLQIGGQENGGDDDDDDCDVAPAA

SEQ ID NO: 28 - protein, 56 aa, Arabidopsis thaliana
MISVSEFVLCIDDNVSGTCMRGKVVISDQAFVYAQSVYVEDGDNDDDDIYDYAPAA

SEQ ID NO: 29 - protein, 56 aa, Arabidopsis thaliana
  MISC_FEATURE (50)..(50): X can be any naturally occuring amino acid
MFSIYKFVLCKWDQVGETFIRGDVTYNNGEFEYPQVAYVENGDDDDDIIXDYAPAA

SEQ ID NO: 30 - protein, 56 aa, Arabidopsis thaliana
MVSVSELVLYVHENVYETCIGVNIANNDQVFEYAQTAFVENGDNDDDVIYDYAPAA

SEQ ID NO: 31 - protein, 53 aa, Arabis alpina
MISVTEFVLCIHENVYDKCNGDGIVNNNGASDSATVENGDNDDDDIYDYAPAA

SEQ ID NO: 32 - protein, 53 aa, Arabis alpina
MISVTEFVLCIDDIVYEKCIAVSGAKSIQASEFTSVENGDNDDDVIYDYAPAA

SEQ ID NO: 33 - protein, 47 aa, Arabis alpina
MAVMSHNKAESRLHESTQACPSPYSVTRAHENGGDDDDDDCDVAPAA

SEQ ID NO: 34 - protein, 56 aa, Arabis alpina
MVFVFHYVLCKYDEVCETFIEGNAIKNCAELEYSQAGYVENGDDDDDNVYDYAPAA

SEQ ID NO: 35 - protein, 49 aa, Brassica rapa
MYLVAHLVIKSFDGDYAVSAEDVVDTSRAAYIENGGDDDDGGYDYAPAA

SEQ ID NO: 36 - protein, 47 aa, Brassica rapa
MAVMSHNKAEGRLYESTQTRLVPYIQTLGQESGGDDDDDDSDVAPAA

SEQ ID NO: 37 - protein, 48 aa, Brassica rapa
MAVMSHDKAEDRLYESAHTRPIPYNSQIVGQESGGDDDDDDSDVAPAA

SEQ ID NO: 38 - protein, 52 aa, Brassica rapa
MFSVSEFLFCTYDNVYGGDITNNDEAVQYAQAVFSENGDDDDDVIYDYAPAA

SEQ ID NO: 39 - protein, 49 aa, Brassica napus
MSFAANLVIINFYCASAVCVEELLDNSLGSYTENGGDDDDSGYDYAPAA

SEQ ID NO: 40 - protein, 57 aa, Brassica napus
MISVREFVFCASNNNICEMCSGGVMANNDKRFEYAQAAYVENGDNDDDVIYDYAPAA

SEQ ID NO: 41 - protein, 52 aa, Brassica napus
MFSVSEFLFCTYDNVYGGDITNNDEAIQYAQAVFSENGDDDDDVIYDYAPAA

SEQ ID NO: 42 - protein, 47 aa, Brassica napus
MAVMSYNKAEGRLYESTQTRPVPYIQTVGQESGGDDDDDDSDVAPAA

SEQ ID NO: 43 - protein, 50 aa, Camelina sativa
MMTFVANLLSKSLDRASSVYVEDVVDSSRVAYGENGGDDDDSGYDYAPAA

SEQ ID NO: 44 - protein, 50 aa, Camelina sativa
MMTFVANLLSKSLDRASSAYVEDVVDSSRVAYGENGGDDDDSGYDYAPAA

SEQ ID NO: 45 - protein, 46 aa, Camelina sativa
MMTLLSKSLDRASSVYVEDVVDSSRVAYGENGGDDDDSGYDYAPAA

SEQ ID NO: 46 - protein, 49 aa, Camelina sativa
MSFVANLVIKSFDRASTVCVEDVVDSFRAAYVENGGDDDDSGYDYAPAA

SEQ ID NO: 47 - protein, 49 aa, Camelina sativa
MSFVANLVIKSFDRASTVCVEDVVDSFRVAYVENGGDDDDSGYDYAPAA

SEQ ID NO: 48 - protein, 49 aa, Camelina sativa
MSFVANLVIKSFDRASTVCVEDVVDSFRVAYVENGGDDDDSGYDYAPVA

SEQ ID NO: 49 - protein, 50 aa, Camelina sativa
MMSSVANLVIKSFDYASTVCVEDVVDSSRAAYVENGGDDDDSGYDYAPAA

SEQ ID NO: 50 - protein, 50 aa, Camelina sativa
MMSSVANLVIKSFDYASTVCLEDVVDSSRAAYVENGGDDDDSGYDYAPAA

SEQ ID NO: 51 - protein, 49 aa, Camelina sativa
MSSVADLVIKSFNHASTVCDEDVVDTFRAAYVESGGDDDDSGYDYAPAA

SEQ ID NO: 52 - protein, 49 aa, Camelina sativa
MSSVADLVIKSFNHASTACDEDVVDSFRAAYVENGGDDDDSGYDYAPAA

SEQ ID NO: 53 - protein, 50 aa, Camelina sativa
MMPSVANLVIKSFEYVSTVCLEDVKDSSRVAYVENGGDDDDSGYDYAPAA

SEQ ID NO: 54 - protein, 50 aa, Camelina sativa
MAVLIVSRNNNGEGRLYESTRTQPIPYLQNGGQENGGDDDDDDCDVAPAA

SEQ ID NO: 55 - protein, 67 aa, Cicer arietinum
MASISMIIAPRCEKHAYGEGDRFCYISTACVELEDYHSGGGDFVSPQVTYNEGDDDDDGGYDYAPAA

SEQ ID NO: 56 - protein, 79 aa, Citrus clementina
MAPMSSSLEGITHGNVHHRDDDSIHVYGCPYYYRNEPFEGDGDDDDDDDDGCDLAPAASMEGDGDDDDDDGGYDYAPAA

SEQ ID NO: 57 - protein, 74 aa, Citrus clementina
MSLVSKSVMPSSSWTWCKKHGDGDDDDDDGYDYAPAACIEGYGDDDDDDGDYDYAPAASMEGDDDGSYDYAPAA

SEQ ID NO: 58 - protein, 76 aa, Citrus clementina
MSSSLEGITHGNVHHRDDDSIHVYGCPYYYRNEPFEGDGDDDDDDDDGCDLAPAASMEGDGDDDDDDGGYDYAPAA

SEQ ID NO: 59 - protein, 57 aa, Cucumis melo
MDVSIFEPVASTMIKNIIAYKDVKCGRQFSTNLTTTIIRREGDDDDDDGCYDYAPAA

SEQ ID NO: 60 - protein, 56 aa, Cucumis sativua
MAPISRLPCVLGLKNLGGDGGHGYREGCDCGYTTLVSMAEGDSDDDDGGYDFAPAA

SEQ ID NO: 61 - protein, 55 aa, Cucumis sativua
MVSTSKSVASMMIKNNVCEDVKCSRSFPIDLTKTIIRREGDDDDDDGCYDYAPAA

SEQ ID NO: 62 - protein, 58 aa, Erythranthe guttata
PNHTHSNSWNLCSKRSELTSEEIRVSPNFGILITQLHDREGDDDDDDDDGGTFVAPAA

SEQ ID NO: 63 - protein, 50 aa, Glycine max
MVVFICKEEYGVPLSNDWAATHEFGHKFCISNEGDDDDDDNGVIDVAPAA

SEQ ID NO: 64 - protein, 50 aa, Glycine max
MVVFICKEEYGVPLSNGWAATHEFGHKFCISNEGDDDDDDNGVIDVAPAA

SEQ ID NO: 65 - protein, 50 aa, Glycine max
MVVFLCKEEYGVLLGNDWAATHEFGHNFCISNEGDDDDDDNGVIDVAPAA

SEQ ID NO: 66 - protein, 51 aa, Glycine max
MVVFVSCTKSGLPLFSKGNDWPATRFPIHQETDDDDDDDDDDGGIDIAPAA

SEQ ID NO: 67 - protein, 50 aa, Glycine max
MVVFVSCTKSGLPLFSKGNDWQATRLSIHQEADDDDDDDDDGGIDIAPAA

SEQ ID NO: 68 - protein, 68 aa, Glycine max
MALTSKAINQECKKHACGNKDGDWYLYYAPTACTEGDDHKGNRDSCFGHIAYMKGDDDGDIYDYAPAA

SEQ ID NO: 69 - protein, 68 aa, Glycine max
MALTSKAINQECKKHACGNKDGDWYLYYAPTACTEGDDHKGNGDSCFGHIAYMKGDDDSDIYDYAPAA

SEQ ID NO: 70 - protein, 64 aa, Glycine max
MAFISMAINLIDCMTHACGNKDNDWYLYAPTACTEGDDPNGDVDSWFAYMEGDDDDGYDYAPAA

SEQ ID NO: 71 - protein, 65 aa, Glycine max
MASMSKAMTPEIKKHACDKKDGVSYHYDPTACAEGDDYNGNINYVFAYMEGDDDDDGGYDYAPAA

SEQ ID NO: 72 - protein, 65 aa, Glycine max
MASMSKAMTPEIKKHACDKKDGVLYHYDPTACAEGDDYNGNINYVFAYMEGDDDDDGGYDYAPAA

SEQ ID NO: 73 - protein, 58 aa, Glycine max
MTPEIKKHACDKKDGVLYHYDPTACAEGDDYNGNINYVFAYMEGDDDDDGGYDYAPAA

SEQ ID NO: 74 - protein, 70 aa, Glycine max
MSFTSKVIALWCKKHGNDDGVDVYDAPAATACIEGNVCNWHGDFVSFVPVALVEGDDDDDDGGYDYAPAA

SEQ ID NO: 75 - protein, 66 aa, Glycine max
MSFTSKVIAPWCKKHGNDDVVDAPAATTFIGGNVCNWHGDFVSFVPIAYMEGDDDDDGGYDYAPAA

SEQ ID NO: 76 - protein, 69 aa, Gossypium hirsutum
MSPFSKVVASSCKKHVDGDYDDNDGFDYAPIACMEGNGDDDDDDDYDYAPAASLDGDDDDDSYDYAPAA

SEQ ID NO: 77 - protein, 72 aa, Jatropha curcas
MSSVLLKAIASSWCNNQNLIIYDDGFDYASVVPSIDGDGGDDDDGDYDYAPAASMEGDDDDDDDDGDCAPAA

SEQ ID NO: 78 - protein, 50 aa, Jatropha curcas
MVIVDSKKLGFFRLVAGEGQAMACFCMSKQNDGDDDDDDDDGGADVAPAA

SEQ ID NO: 79 - protein, 48 aa, Lotus japonicus
MVVLVCKESRLPKFFMAPPELQSFCIQNESDSDDDDDGDNDIDIAPAA

SEQ ID NO: 80 - protein, 50 aa, Medicago truncatula
MSSISNVVAPWCKKHGNDHDGCVVWYDYPPTVCDEGDDDDDGGYDYAPAA

SEQ ID NO: 81 - protein, 62 aa, Medicago truncatula
MVFISMVIALNCKQHAYGEGEVDWFGCTSVSCIEEDYHNTDHDSYWEGDDDDDGGYDYAPAA

SEQ ID NO: 82 - protein, 60 aa, Medicago truncatula
MASISMVIALNCKQHAYGEGNWFDYTSVSCIEEDYHNGDRDSYQEGDDDDDGGYDYAPAA

SEQ ID NO: 83 - protein, 62 aa, Medicago truncatula
MASIFTVIAPLCKQNACGEGNGDWFGYTSVSCIVEDYRNGDQDSYKEGDDDDDGGYDYAPAA

SEQ ID NO: 84 - protein, 63 aa, Medicago truncatula
MTFISTVIAPKCKQYAYNGEGDGDWFGYTCVSCIEEDYTNGDRNLYREGDDDDDGGYDYAPAA

SEQ ID NO: 85 - protein, 62 aa, Medicago truncatula
MASISLAIIPKCEQHGYGEGNGDWISYTCVSCIEENYHNGDRDSCKEGDDDDDGGYDYAPAA

SEQ ID NO: 86 - protein, 62 aa, Medicago truncatula
MASISLAITPKCKHHGYSEGNGDWFGYTSVSCIKEDNRNGDRDSCKEGDDDDDGGYDYAPTA

SEQ ID NO: 87 - protein, 62 aa, Medicago truncatula
MASISMAITPKCKEHGYDEGNGDWFGYTYVSCIEEDYRNGDRDSYMEGDDDDDGGYDYAPAA

SEQ ID NO: 88 - protein, 58 aa, Medicago truncatula
MASISMVIAPKCKQHAFNGEGDCDWFGYTNISCIEEDYYNGEGDDDDDDGGYDYAPAA

SEQ ID NO: 89 - protein, 59 aa, Medicago truncatula
MASISIAIATKSIKNVYDGGEWFGYASVACIEDYHIGDVDSYKEGDDDDDGGYDYAPAA

SEQ ID NO: 90 - protein, 61 aa, Medicago truncatula
MASISIAIATRSIKHVYDEDEWFGYAASVACIEDYHTEDVDSSYKEGDDDDDGGYDYAPAA

SEQ ID NO: 91 - protein, 58 aa, Medicago truncatula
MASISIPTRSIKNACGEGEWFGFASVSCNDEDYHTGDVDSYREGDDDDDGDYDYAPAA

SEQ ID NO: 92 - protein, 60 aa, Medicago truncatula
MAFISISIATRSFQNACDEGEWFCYASVGCIEQDNHIGEEDSYREGDDDDDGGYDYAPAA

SEQ ID NO: 93 - protein, 70 aa, Medicago truncatula
MTSISMVNISPKCNHAAYGECDGDWFGYASTICIKGNYYFRNEDSGSAHLIAYIEGDDDDDGGYDYAPAA

SEQ ID NO: 94 - protein, 70 aa, Medicago truncatula
MASISIVNTAPKCNHAAYGECDVDSFGYASTVCIKGNYYIRNEDSGSADLVTYMEGDDDDDGGYDYAPAA

SEQ ID NO: 95 - protein, 60 aa, Morus notabilis
MPHITFMNMVTARNGKDGDNGDHCYDHYFQYYNLSAPGEGDGDDDNDDDDDSGYDYAPAA

SEQ ID NO: 96 - protein, 79 aa, Morus notabilis
MFPAVDKLIYSESLSKKREDGGNHEDGNDGISRRYAPTQVMENIYGASDRDYNYLPNATMDGDDDDDDDDSSYDYAPAA

SEQ ID NO: 97 - protein, 62 aa, Morus notabilis
MSPVSEKNVAILAIMLCKKQNITYGDVTNGDFEYNLDPVTRIEGDDDDDDDDDGGYDYAPAA

SEQ ID NO: 98 - protein, 54 aa, Oryza sativa
MAPVSEASPLVHQDGGIIASFAVYAGAPCCSARGRMAETDGDDDDDDYDCAPAA

SEQ ID NO: 99 - protein, 43 aa, Oryza sativa
MAIAKSECERLAWALLLESNLLVGNRRSNGDDDDDDVDVAPAA

SEQ ID NO: 100 - protein, 49 aa, Pisum sativum
MVIITSMESTLPMFLMDNHLSATEFICFCNQSKSEGDDDDGDIDIAPAA

SEQ ID NO: 101 - protein, 66 aa, Phaseolus vulgaris
MAFTSKLIAPCCNNHHALHQNHHAPPTFIEESVFNGHGDSVSFVPAASMEGDDDDDDGSYDYAPAA

SEQ ID NO: 102 - protein, 49 aa, Phaseolus vulgaris
MLLFISCTKSGLPLFIKGNDWPETIFPLHHEGDDDDDDDDGGIDVAPAA

SEQ ID NO: 103 - protein, 44 aa, Phaseolus vulgaris
MMFFVCKEYGLVLSNDWPATHDFHNFHEGDDDDDNGVIDVAPAA

SEQ ID NO: 104 - protein, 71 aa, Populus trichocarpa
MSPLSKTIIAFCTIHRHADGDDDGEYGYDYAPAACMEGDGDDDDSDYDYAPAAPMEGDDDDDGDYDYAPAA

SEQ ID NO: 105 - protein, 72 aa, Ricinus communis
MSLFAISKAITISCCNKLADDCDNGDGCYFAPPPCIEGDGDDDDGDYDYAPAASSEGDDDDDDDGYDCAPAA

SEQ ID NO: 106 - protein, 84 aa, Ricinus communis
MVHMAFLTARSSGKCSSTNLADQNKDIDVLFGYNEFSMEAPLIEDDGDGDDDDDDGGYDFAPAATLEGDGDDDDDGDYDYAPAA

SEQ ID NO: 107 - protein, 55 aa, Ricinus communis
MSPLSKVIASWCNKPVVEEEMGNDDDRVYEYAPATSTEGDDDDDDDDDKDCAPAA

SEQ ID NO: 108 - protein, 45 aa, Solanum lycopersicum
MTEIYSFHLYNKIELTRPVISFCLNRELENDDDDDDDGKKVAPAA

SEQ ID NO: 109 - protein, 50 aa, Solanum lycopersicum
MVIVRGNTTPFLPGRIEARPVINFGLNREFEADDDDDDDDDDGKKVAPAA

SEQ ID NO: 110 - protein, 51 aa, Solanum lycopersicum
MVIVRGNTSRFHPYEIEARLVISFYLNRELENDVDDDDDDDDDGAKVAPAA

SEQ ID NO: 111 - protein, 66 aa, Solanum lycopersicum
MSQISTILMNSICNFNHDVGSHVYESRSMMDRHATGCIYVSATWFEDDGDDDDDDDDADYDYAPAA

SEQ ID NO: 112 - protein, 78 aa, Solanum lycopersicum
MSTIFMIIGFEKRRSCADGDYDYTSAASLEGDDDDGDYDYAPAASLEGNDDDGDYDYAPAASLEGDDDDGDYDYAPAA

SEQ ID NO: 113 - protein, 83 aa, Solanum lycopersicum
MFGIFKIIGFEKIRRSCLDGDDDGDYDYAPAACLKRNGDDDGDYDYAPAAFLEGDDDDRDYDCAPAATIDGDDDGDYDYAPAA

SEQ ID NO: 114 - protein, 86 aa, Solanum lycopersicum
MSGILKIIGFEKIRRSCLDGDDDSDYDYAPAACLERDGDDDGDYDYAPAASLEGDDDDRDYDYVPAASLEGDDDGDYDYAPSGCMK

SEQ ID NO: 115 - protein, 83 aa, Solanum lycopersicum
MSGIFKIIGFEKIKRSCLDGDDDGDYDFAPAACLERDGDDDGDYDYAPAASLEGDDDDRDYDYVPAASLEGDDDGDYDYAPAA

SEQ ID NO: 116 - protein, 84 aa, Solanum lycopersicum
MSSIFKIIGFQKRRSCSDGDDDGDYDYAPSACLEGGGDGDDGDYDYTPAASLEGDCNDQDYDYAPAVSFEGHDVDGDYDYAPAA

SEQ ID NO: 117 - protein, 81 aa, Solanum tuberosum
MAGKSGRKVVRGVSKSSKAVYFWKIRHGCGIIFGKSKKYKSCSYGSEDDDDDEHDYAPATYLERDDDDDDGNYDYAPAALT

SEQ ID NO: 118 - protein, 55 aa, Solanum tuberosum
MVVIMNANNKKVSLGCPDKFMATEAKLGSTICIDKECEADDDYNDDDASKIAPAA

SEQ ID NO: 119 - protein, 83 aa, Solanum tuberosum
MSGTFKIIGFQKRRSSSDGDDEGYNYAPVTCLEGDGDDNDAYYDYASAPFLEGDDDDGDYDYAPATSLEGEDNDGDYDYAPAA

SEQ ID NO: 120 - protein, 86 aa, Solanum tuberosum
MSTIFKIIGFQKRRSCSDGDDDSDDGYDYAPAACLEGDGDDNDGDYDYAPAASLEGDDDDGDYDYAPAASLEGDDDDGDYDYAPAA

SEQ ID NO: 121 - protein, 78 aa, Solanum tuberosum
MSTIFKIIGFEKRRSCPDGNYNYTLVASLEGDDDDGDYDYAPAASLEGDDDDGDYDYAPAASLEGDDDDGNYDYAPAA

SEQ ID NO: 122 - protein, 82 aa, S. tuberosum
MSSIFKIIGFQNKRSYSDGDDDGDYDFAPAAFLEGDDDDGDYDYAPAASLNGDDDDGDYDYVPAASLDGDDDDGDYDYAPAA

SEQ ID NO: 123 - protein, 82 aa, Solanum tuberosum
MSSIFKIIGFHNRRSYSDGDDDGDYDYAPAAYLEGDDDDEDYDYAPAASLNGDDDDGDYDYAPAASLEGDDDDGDYDYAPAA

SEQ ID NO: 124 - protein, 82 aa, Solanum tuberosum
MSSIFKIIGFQNRRSYSDGDDDGDYDYAPTAYLEGDDDDGDYDYAPVASLNGDDDDGDYDYAPATSLEGDDDDGDYDYAPAA

SEQ ID NO: 125 - protein, 82 aa, Solanum tuberosum
MSSIFKIIGFEKRRSCLDGDDDGDYDYAPAACLERDGDDDGDYDYAPAASLEGDDDDRDYDYAPAASLEGDDDGDYDYAPAA

SEQ ID NO: 126 - protein, 84 aa, Solanum tuberosum
MSSIFKIIGFQKRRLCLDGDDDGDYDYAPAACLEGGGDRDDGDYYYALAASLEGDDDDRDYDYAPAASLGGDGDDGDYDYAPVA

SEQ ID NO: 127 - protein, 78 aa, Solanum tuberosum
MSQISTILMNSICNLTFFDYHVERGNHDIGSHVYESTSMMDRHVIGCIYVSATWFEDDGDDDDDDDDDDADYDYAPAA

SEQ ID NO: 128 - protein, 83 aa, Solanum tuberosum
MSEIFTIIGFEKIRRSCLDGDDDGDYDYAPASCLERDGDDDGDYDYAPAASLEGDDDDRDYDYVPAASLEGDDDGDYDYAPAA

SEQ ID NO: 129 - protein, 83 aa, Solanum tuberosum
MSSGIFKILGFEKRRLCSYGDDDGDYVYAPAACLNRDGDDDGEYDYAPAASLEGDDDDRDYDYAPATNLEGDDDRDYDYASAA

SEQ ID NO: 130 - protein, 79 aa, Theobroma cacao
MSSSKCIMHDEDNIKKIGSSSKNIMNDDVHDDKGRRDGYVSNSKSLVQGGNSYTHVPSASVDGDGDDDDDDDYDFAPAA

SEQ ID NO: 131 - protein, 56 aa, Triticum urartu
MAPASKVMSHVVQDGGIADYAVYAAAPCDAWCGGRHRKAESDGDDDDDDYDCAPAA

SEQ ID NO: 132 - protein, 57 aa, Triticum urartu
MAPASKIMSHIVVQDGGIAAYAVYAAAPCDAWCGGRHRKAESDGDDDDDDYDCAPAA

SEQ ID NO: 133 - protein, 55 aa, Triticum urartu
MAPASKAMSHIVQDGGIATYAVYAAPCDAWCGGRHRKAETDGDDDDDDYDCAPAA

SEQ ID NO: 134 - protein, 65 aa, Vitis vinifera
MSSISMAIDSQSMMHDGHVRGEHDKHGHVYCTNDNGGCYYALTAPREGDGDDDDGDGGYDFAPAA

SEQ ID NO: 135 - protein, 57 aa, Zea mays
MAPVSSEAASYLVLIKGGSIAASSRAVYPWDGCSARGRMTETDSDDDDDDYDCAPAA

SEQ ID NO: 136 - protein, 50 aa, Artificial Sequence
  consensus sequence shown in Fig. 2F
MXXXXXNXAXXRXXXASTXXXXXXXXXSRXXXXENGGDDDDXXXDXAPAA

SEQ ID NO: 137 - protein, 41 aa, Artificial Sequence
  IMA1 deletion 1
MMSFVAASTVYVEDVVDSSRVAYSENGGDDDDSGYDYAPAA

SEQ ID NO: 138 - protein, 43 aa, Artificial Sequence
  IMA1 deletion 2
MMSFVANLAIKRFDHASTVYVEDVVDENGGDDDDSGYDYAPAA

SEQ ID NO: 139 - protein, 45 aa, Artificial Sequence
  IMA1 deletion 3
MMSFVANLAIKRFDHASTVYVEDVVDSSRVAYSENGSGYDYAPAA

SEQ ID NO: 140 - DNA, 20 bp, Artificial Sequence
  primer
tccccagttagctgatttcg

SEQ ID NO: 141 - DNA, 20 bp, Artificial Sequence
  primer
ctttgccgatcatccttagc

SEQ ID NO: 142 - DNA, 18 bp, Artificial Sequence
  primer
tgcgtcaacaaagctaaa

SEQ ID NO: 143 - DNA, 20 bp, Artificial Sequence
  primer
tctggttggaggaacgaaac

SEQ ID NO: 144 - DNA, 26 bp, Artificial Sequence
  primer
gatcgaaaaaagcaataacggtggtt

SEQ ID NO: 145 - DNA, 25 bp, Artificial Sequence
  primer
gatgtggcaaccacttggttcgata

SEQ ID NO: 146 - DNA, 22 bp, Artificial Sequence
  primer
cagtcacaagcgaagaaactca

SEQ ID NO: 147 - DNA, 24 bp, Artificial Sequence
  primer
cttgtaaagagatggagcaacacc

SEQ ID NO: 148 - DNA, 23 bp, Artificial Sequence
  primer
atgtcttttgtcgcaaacttggc

SEQ ID NO: 149 - DNA, 25 bp, Artificial Sequence
  primer
caccaccattctcactatatgccac

SEQ ID NO: 150 - DNA, 24 bp, Artificial Sequence
  primer
gacggtaccacagacttatgaagt

SEQ ID NO: 151 - DNA, 25 bp, Artificial Sequence
  primer
taagctctttgaaaccgtttcagga

SEQ ID NO: 152 - DNA, 24 bp, Artificial Sequence
  primer
gacttatggagctgttacagcggt

SEQ ID NO: 153 - DNA, 25 bp, Artificial Sequence
  primer
cttcaagcttcgagaaaccgtcgca

SEQ ID NO: 154 - DNA, 26 bp, Artificial Sequence
  primer
tgattgtaatttaggaggaaacaaaa

SEQ ID NO: 155 - DNA, 25 bp, Artificial Sequence
  primer
tcaatccacaagtaaacatctatgg

SEQ ID NO: 156 - DNA, 22 bp, Artificial Sequence
  primer
gctgttcgtggtgttgagatgc

SEQ ID NO: 157 - DNA, 22 bp, Artificial Sequence
  primer
aggctctgaggtgaggaagtct

SEQ ID NO: 158 - DNA, 22 bp, Artificial Sequence
  primer
tgagtcacaacaacgcagaagg

SEQ ID NO: 159 - DNA, 22 bp, Artificial Sequence
  primer
ggccaagtctgagttgattcgt

SEQ ID NO: 160 - DNA, 22 bp, Artificial Sequence
  primer
tgagtcacaacaacgcagaagg

SEQ ID NO: 161 - DNA, 22 bp, Artificial Sequence
  primer
ggccaagtctgagttgattcgt

SEQ ID NO: 162 - DNA, 19 bp, Artificial Sequence
  primer
gagcccaagtttttgaaga

SEQ ID NO: 163 - DNA, 19 bp, Artificial Sequence
  primer
ctaacagcgaaacgtccca

SEQ ID NO: 164 - DNA, 28 bp, Artificial Sequence
  primer
gcgggatcccatcaacatttgaagctca

SEQ ID NO: 165 - DNA, 29 bp, Artificial Sequence
  primer
cgcggatccggaaactagcaatattataa

SEQ ID NO: 166 - DNA, 33 bp, Artificial Sequence
  primer
aaatctagaatgatgtcttttgtcgcaaacttg

SEQ ID NO: 167 - DNA, 30 bp, Artificial Sequence
  primer
aaagagctctcacgcagcaggagcataatc

SEQ ID NO: 168 - DNA, 35 bp, Artificial Sequence
  primer
aaatctagaatggcagtggtgagtcacaacaacgc

SEQ ID NO: 169 - DNA, 28 bp, Artificial Sequence
  primer
aaagagctctcaagccgccggtgcaacg

SEQ ID NO: 170 - DNA, 18 bp, Artificial Sequence
  primer
gcttccaccgtgtatgtt

SEQ ID NO: 171 - DNA, 18 bp, Artificial Sequence
  primer
tgcgacaaaagacatcat

SEQ ID NO: 172 - DNA, 22 bp, Artificial Sequence
  primer
gagaatggtggtgatgacgatg

SEQ ID NO: 173 - DNA, 26 bp, Artificial Sequence
  primer
atctaccacatcttcaacatacacgg

SEQ ID NO: 174 - DNA, 23 bp, Artificial Sequence
  primer
agtggctatgattatgctcctgc

SEQ ID NO: 175 - DNA, 29 bp, Artificial Sequence
  primer
accattctcactatatgccactcgagaac

SEQ ID NO: 176 - DNA, 21 bp, Artificial Sequence
  primer
gttgtaaaacgacggccagtc

SEQ ID NO: 177 - DNA, 21 bp, Artificial Sequence
  primer
tgccaggaaacagctatgacc

SEQ ID NO: 178 - DNA, 40 bp, Artificial Sequence
  primer
gattactaataggagacaatcattctctcttttgtattcc

SEQ ID NO: 179 - DNA, 40 bp, Artificial Sequence
  primer
gaatgattgtctcctattagtaatcaaagagaatcaatga

SEQ ID NO: 180 - DNA, 40 bp, Artificial Sequence
  primer
gaataattgtctccttttagtattcacaggtcgtgatatg

SEQ ID NO: 181 - DNA, 40 bp, Artificial Sequence
  primer
gaatactaaaaggagacaattattctacatatatattcct

SEQ ID NO: 182 - DNA, 25 bp, Artificial Sequence
  primer
ctgcaaggcgattaagttgggtaac

SEQ ID NO: 183 - DNA, 28 bp, Artificial Sequence
  primer
gcggataacaatttcacacaggaaacag

(19) United States
(12) Patent Application Publication
(10) Pub. No.: US 2015/0315250 A1
(43) Pub. Date: Nov. 5, 2015

(54) TRANSGENIC PLANTS WITH INCREASED TRACE ELEMENT CONTENTS AND METHODS FOR PRODUCING THE SAME

(71) Applicant: Academia Sinica, Taipei (TW)

(72) Inventors: Wolfgang Schmidt, Taipei City (TW); Ping Lan, Nanjing (CN); Louis Grillet, Taipei City (TW)

(73) Assignee: Academia Sinica, Taipei (TW)

(21) Appl. No.: 14/702,130

(22) Filed: May 1, 2015

Related U.S. Application Data
(60) Provisional application No. 61/987,638, filed on May 2, 2014.

(51) Int. Cl. C07K 14/415; A23L 1/304; A61K 36/31; A23L 1/212; C12N 15/82; A61K 33/26

(52) U.S. Cl. CPC C07K 14/415; C12N 15/8243; A61K 33/26; A61K 36/31; A23L 1/212; A23L 1/304; A23V 2002/00

(57) ABSTRACT

The present invention relates to a transgenic plant with increased trace element contents and a method for producing the same. In particular, the transgenic plant is incorporated by a polynucleotide encoding an iron-regulated protein 1 (IRP1/IMA1) or IRP1-like (IRL/IMA3) polypeptide, which facilitate uptake and circulation of the trace elements into the plant. Also provided is a method for treating trace element deficiency by administrating to a subject in need a composition comprising a transgenic plant as described or an edible tissue or part thereof.

### RELATED APPLICATION

The present application claims priority under 35 U.S.C. §119(e) to U.S. Provisional Patent Application No. 61/987,638, filed May 2, 2014, the content of which is herein incorporated by reference in its entirety.

### TECHNOLOGY FIELD

The present invention relates to a transgenic plant with increased trace element contents and a method for producing the same.

### BACKGROUND OF THE INVENTION

Deficiency of trace element nutrition such as iron (Fe), zinc (Zn) and manganese (Mn) is a global problem. There are several strategies that have been used to combat this problem, one of which is genetically modifying plants in which the trace element nutrition is increased. In such manner, trace elements of a subject who consumes these plants may be improved.

Although iron (Fe) is one of the most abundant elements on earth, Fe deficiency is the most widespread nutritional disorder in human populations. Iron deficiency-induced anemia (IDA) caused by insufficient dietary Fe intake particularly in areas where Fe supply depends mainly or entirely on plants affects more than billion people worldwide. Increasing the bio-available Fe levels in soils by applying Fe fertilizers is costly, not sustainable, and cannot be directed to desired plant parts. Improving the acquisition of Fe and its transport to edible plant parts is thus mandatory to combat IDA.

Plants have evolved multifaceted strategies to acquire Fe from soils (1). Graminaceous species take up Fe after secretion of phytosiderophores (PS) from the mugineic acid family that bind Fe with high affinity by TOM1 and subsequent uptake of the (ferric) Fe-PS complex by YSL transporters. Arabidopsis and all non-grass crop species employ a reduction-based Fe acquisition strategy, in which Fe is first reduced by the oxidoreductase AtFRO2. Ferrous Fe is then transported across the plasma membrane by AtIRT1 (1,2). The two Fe acquisition strategies were thought to be mutually exclusive (4). However, rice (Oryza sativa) possesses a Fe²⁺ uptake system (5) and Arabidopsis secretes Fe-binding coumarins resembling the PS-system of grasses (6-8), indicating that the two Fe acquisition strategies can comprise common components.

In Arabidopsis, the bHLH-type transcription factors AtPYE and AtFIT control non-overlapping subsets of genes involved in the acquisition and cellular homeostasis of Fe (9). AtFIT acts as heterodimer with the 1b subgroup bHLH transcription factors AtbHLH038, AtbHLH039, AtbHLH100 and AtbHLH101 (10,11). In rice (Oryza sativa) OsIRO2, an ortholog of AtbHLH100/101, regulates the Fe-PS transporter OsYSL15, but not the uptake of Fe²⁺ via OsIRT1 (12). The genes encoding AtbHLH038/39/100/101 and OsIRO2 are Fe-responsive, suggesting upstream regulatory components. Similar to animals, Fe sensing in plants occurs via direct binding of Fe to regulatory proteins, OsIDEF1/OsHRZs in rice and AtBTS in Arabidopsis (13,14).

There is a need to produce a transgenic plant with increased trace element contents by which the problem of trace element deficiency can be solved.

### SUMMARY OF THE INVENTION

We report here a novel family of peptides that share a short C-terminal amino acid sequence motif conserved in numerous, highly diverse peptides across angiosperms. We named this peptide sequence IRON MAN (IMA), referring to its ability to trigger iron and manganese accumulation through activation of iron uptake genes. It is unexpectedly found that IMA is critical in iron deficiency signaling in plants, acting early in the cascade that controls uptake, transport and cellular homeostasis of iron, and plants overexpressing IMA peptides exhibit an increased level of one or more of the trace elements, such as Fe, Zn and/or Mn, which are of improved nutritive values to animals, particularly in respect of overcoming the problems of trace element deficiencies. It is also found that the C-terminal motif is critical for the function of IMA peptides since deletions in the C-terminal motif of recombinant IMA peptides can completely abolish their function. Manipulating the expression of IMA peptides represents a novel strategy for iron bio-fortification in crops.

Particularly, in a first aspect, the present invention provides a transgenic plant transformed with a recombinant polynucleotide comprising a nucleotide sequence encoding an iron-regulated polypeptide (i.e. IMA peptide as used herein), operatively linked to an expression control sequence,

wherein the iron-regulated polypeptide comprises a C-terminal motif comprising from N-terminal to C-terminal
a first domain of GDDDD (SEQ ID NO: 1), and
a second domain of DXAPAA (SEQ ID NO: 2),
in which the first domain and said second domain are joined by a peptide spacer of 10 or less amino acid residues,

wherein the iron-regulated polypeptide comprises a total of 20 to 100 amino acid residues in length.

In some embodiments, the iron-regulated polypeptide can increase ferric reduction activity or can activate one or more transcriptional factors for Fe homeostasis in plants, selected from the group consisting of AtbHLH38, AtbHLH39, AtFIT and any combinations thereof.

In some embodiments, the transgenic plant overexpresses the iron-regulated polypeptide and has a content of a trace element higher than that present in a control plant, where the trace element is selected from the group consisting of iron (Fe), zinc (Zn) and manganese (Mn).

In some embodiments, the iron-regulated polypeptide comprises a total of 20 to 90, 25 to 85 or 45 to 75 amino acid residues in length.

In some embodiments, the peptide spacer between the first domain and the second domain of the iron-regulated polypeptide has a total of 1 to 6 or 1 to 3 any amino acid residues.

In some embodiments, the C-terminal motif comprises the amino acid sequence selected from the group consisting of: SEQ ID NOs: 3, 4, 5, 6, 7, 8 and 9.

In some embodiments, the iron-regulated polypeptide comprises the amino acid sequence selected from the group consisting of: SEQ ID NOs: 25, 26, 27, 63, 65, 74, 75, 98 and 99.

In a second aspect, the present invention provides a plant tissue or part or plant cell of a transgenic plant as described herein.

In a third aspect, the present invention provides a method for producing a transgenic plant, comprising (a) transforming a plant cell with a recombinant polynucleotide comprising a nucleotide sequence encoding the iron-regulated polypeptide as described herein, and (b) growing the recombinant plant cell obtained in (a) to generate a transgenic plant.

In a fourth aspect, the present invention provides a method for biofortification comprising growing a transgenic plant as described herein or its seed or other propagating materials under a condition to express the iron-regulated polypeptide, sufficient for a content of a trace element to increase in the transgenic plant, wherein the trace element is selected from the group consisting of iron (Fe), zinc (Zn) and manganese (Mn).

In a fifth aspect, the present invention provides a plant product made from a transgenic plant or a plant tissue, plant part or plant cell thereof. The present invention also provides a composition comprising such plant product, which can be made as a nutritional supplement or a pharmaceutical composition for use in supplementing trace element in a subject in need.

In a sixth aspect, the present invention provides a method of supplementing a trace element in a subject, comprising administering an effective amount of a transgenic plant, a plant product made therefrom or a composition comprising the plant product as described herein.

In some embodiments, the method of the invention is effective in treating symptoms or diseases caused by trace element deficiency, including iron-deficiency, zinc-deficiency or manganese-deficiency. In certain examples, the trace element deficiency is iron-deficiency, which causes anemia.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows identification of the G-D-D-D-D-spacer-D-x-A-P-A-A sequence motif. (A) Sequence logo of the motif identified using the MEME suite (24). (B) Diagram showing the locations of the sequence motifs.

FIG. 2 shows the G-D-D-D-D-spacer-D-x-A-P-A-A motif is critical for the function of IMA peptides. (A) Accumulation of Fe in 35Spro::IMA1_{cDNA} lines. Ectopic expression of AtIMA1 caused leaf bronzing (left panel). Perls' staining revealed high Fe concentrations particularly in leaf veins (middle left), in the root stele (middle right) and in embryos (right panel) in comparison with the wild type. (B) Transition metal concentration in transgenic plants carrying the 35Spro::IMA1_{cDNA} construct in Arabidopsis leaves (left) and seeds (right). (C) Ferric reduction activity of embryos. Reduction activity was determined with three batches of 30 embryos in three separate runs. Error bars show standard errors of the mean. (D) Amino acid sequence alignment of peptides harboring the IMA motif encoded by Fe-responsive genes of Arabidopsis roots/leaves (16,18), tomato (designated SlIMA1; 19), rice roots/leaves (designated OsIMA1 and OsIMA2; 15) and soybean (designated GmIMA1-5; 20). (E) Analysis of the active domain of IMA1. Ferric reduction activity of transgenic plants constitutively expressing the AtIMA1 or AtIMA3 coding sequence (35Spro::IMA1_{ORF}) or chimeric AtIMA1 genes harboring deletions in the variable region (35Spro::IMA1_{ORF}Δ1 and 35Spro::IMA1_{ORF}Δ2) or in the conserved C-terminus of the peptide (35Spro::IMA1_{ORF}Δ3). (F) Alignment of the amino acid sequences of Arabidopsis IMA1 and IMA3. (G) Alignment of the amino acid sequences of Arabidopsis IMA1, the chimeric IMA1Δ1, IMA1Δ2 and IMA1Δ3, and IMA1 expression levels in the lines overexpressing IMA1_{ORF}, IMA3_{ORF}, and chimeric IMA1 harboring deletions.

FIG. 3 shows characterization of AtIMA1 expression pattern, subcellular localization, and effects of AtIMA1 overexpression on Fe homeostasis genes. (A) Relative AtIMA1 transcript abundance in different plant parts. (B) Expression changes of AtIMA1 in response to phosphate and Fe deficiency. (C) Intracellular localization of AtIMA1 determined by the expression of a 35Spro::IMA1:YFP construct in Arabidopsis protoplasts. YFP signals were confined to nuclei and to the cytoplasm. (D) Effect of overexpression of AtIMA1 on transcript profiles determined by quantitative RT-PCR in roots and (E) microarray analysis using the ATH1 gene chip in leaves. Numbers refer to genes that are more than 1.5-fold induced with P<0.05.

FIG. 4 shows effect of heterologous expression of AtIMA1 in tomato plants. (A) Transition metal concentration in fruits. (B) histochemical iron detection in stem cross-sections of wild-type (top) and 35Spro::IMA1_{cDNA} plants (bottom). Error bars denote standard error of the mean.

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a component” includes a plurality of such components and equivalents thereof known to those skilled in the art.

The term “polynucleotide” or “nucleic acid” refers to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides.

Polynucleotides can be synthesized, for example, using an automated DNA synthesizer. The term “nucleic acid” typically refers to large polynucleotides. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

The term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′.”

The term “encoding” refers to the inherent property of specific sequences of nucleotides in a polynucleotide (e.g., a gene, a cDNA, or an mRNA) to serve as templates for synthesis of other polymers and macromolecules in biological processes having either a defined sequence of nucleotides (i.e., rRNA, tRNA and mRNA) or a defined sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “recombinant polypeptide” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence or maintaining the given nucleotide sequence for replicating it, manipulating it or transferring it between different locations (e.g., between different organisms). Vectors can be introduced into a suitable host cell for the above mentioned purposes.

As used herein, the term “operably linked” may mean that a polynucleotide is linked to an expression control sequence in such a manner to enable expression of the polynucleotide when a proper molecule (such as a transcriptional factor) is bound to the expression control sequence.

As used herein, the term “expression control sequence” or “regulatory sequence” means a DNA sequence that regulates the expression of the operably linked nucleic acid sequence in a certain host cell.

Examples of vectors include, but are not limited to, plasmids, cosmids, phages, YACs or PACs. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprises, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, an operator sequence, a secretion signal sequence (e.g., a-mating factor signal) and other control sequence (e.g., Shine-Dalgarno sequences and termination sequences). Preferably, vectors may further contain a marker sequence (e.g., an antibiotic resistant marker sequence) for the subsequent screening procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes, but is not limited to, a His-tag fused polypeptide and a GST fused polypeptide.

Where the expression vector is constructed for a plant cell, several suitable promoters known in the art may be used, including but not limited to the Figwort mosaic virus 35S promoter, the cauliflower mosaic virus (CaMV) 35S promoter, the commelina yellow mottle virus promoter, the rice cytosolic triosephosphate isomerase (TPI) promoter, the rice actin 1 (Act1) gene promoter, the ubiquitin (Ubi) promoter, the rice amylase gene promoter, the adenine phosphoribosyltransferase (APRT) promoter of Arabidopsis, the mannopine synthase and octopine synthase promoters.

To prepare a transgenic plant, it is preferable that the expression vector as used herein carries one or more selection markers for selection of the transformed plants, for example, genes conferring the resistance to antibiotics such as hygromycin, ampicillin, gentamicine, chloramphenicol, streptomycin, kanamycin, neomycin, geneticin and tetracycline, URA3 gene, genes conferring the resistance to any other toxic compound such as certain metal ions or herbicide, such as glufosinate or bialaphos.

As used herein, the term “transgenic plant” or “transgenic line” refers to a plant that contains a recombinant nucleotide sequence. The transgenic plant can be grown from a recombinant cell. The term “plant” as used herein can comprise any material of the plant, including a cell of the plant (including callus), any part or organ of the plant and the progeny.

A variety of procedures that can be used to engineer a stable transgenic plant are available in this art. In one embodiment of the present invention, the transgenic plant is produced by transforming a tissue of a plant, such as a protoplast or leaf-disc of the plant, with a recombinant Agrobacterium cell comprising a polynucleotide encoding an iron-regulated polypeptide as described herein and generating a whole plant from the transformed plant tissue. In another embodiment, a polynucleotide encoding a desired protein can be introduced into a plant via gene gun technology, particularly if transformation with a recombinant Agrobacterium cell is not efficient in the plant.

The term “polypeptide” or “peptide” refers to a polymer composed of amino acid residues linked via peptide bonds.

To determine the percent identity of two amino acid sequences, the sequences are aligned for optimal comparison purposes (e.g., gaps can be introduced in the sequence of a first amino acid sequence for optimal alignment with a second amino acid sequence). In calculating percent identity, typically exact matches are counted. The determination of percent homology or identity between two sequences can be accomplished using a mathematical algorithm known in the art, such as BLAST and Gapped BLAST programs, the NBLAST and XBLAST programs, or the ALIGN program.

In this invention, it is unexpectedly found that a novel family of iron-regulated polypeptides that share only a short C-terminal amino acid sequence motif conserved in numerous, highly diverse peptides across angiosperms, can trigger iron, zinc and manganese accumulation through activation of iron uptake genes, and a plant overexpressing such iron-regulated polypeptide (also named IMA peptide herein) exhibit an increased level of one or more of the trace elements, such as Fe, Zn and/or Mn. Manipulating the expression of IMA peptides represents a novel strategy for iron bio-fortification in edible plants, such as crops or fruit trees.

Therefore, in one aspect, the present invention provides a transgenic plant transformed with a recombinant polynucleotide comprising a nucleotide sequence encoding an iron-regulated polypeptide as described herein, operatively linked to an expression control sequence.

According to the present invention, the iron-regulated polypeptide as described herein comprises a C-terminal motif comprising from N-terminal to C-terminal

a first domain of GDDDD (SEQ ID NO: 1), and

a second domain of DXAPAA (SEQ ID NO: 2),

in which the first domain and said second domain are joined by a peptide spacer of 10 or less amino acid residues, and wherein the iron-regulated polypeptide comprises a total of 20 to 100 amino acid residues in length.

In some certain embodiments, the iron-regulated polypeptide of the invention comprises a total of 20 to 90, 25 to 85 or 45 to 75 amino acid residues in length.

As used herein, the term “C-terminal motif” of the iron-regulated polypeptide means that this motif is located closer to the C-terminus, but farer to the N-terminus, of the iron-regulated polypeptide, preferably the iron-regulated polypeptide is ended with said “C-terminal motif.” Typically, in a linear amino acid sequence, the C-terminal motif is conventionally written to the right.

In some particular embodiments, the first domain and said second domain of the iron-regulated polypeptide of the invention are joined by a peptide spacer having a total of 1 to 6 or 1 to 3 any amino acid residues.

In certain embodiments, the C-terminal motif of the iron-regulated polypeptide of the invention comprises the amino acid sequence selected from the group consisting

(SEQ ID NO: 3)
GDDDDSGYDYAPAA;
(SEQ ID NO: 4)
GDDDDDDCDVAPAA;
(SEQ ID NO: 5)
GDDDDDDNGVIDVAPAA;
(SEQ ID NO: 6)
GDDDDDGGYDYAPAA;
(SEQ ID NO: 7)
GDDDDDDGGYDYAPAA;
(SEQ ID NO: 8)
GDDDDDDYDCAPAA;
and
(SEQ ID NO: 9)
GDDDDDDVDVAPAA.

In certain embodiments, the iron-regulated polypeptide of the invention comprises the amino acid sequence selected from the group consisting of: SEQ ID NOs: 25, 26, 27, 63, 65, 74, 75, 98 and 99.

(SEQ ID NO: 25)
MMSFVANLAIKRFDHASTVYVEDVVDSSRVAYSENGGDDDDSGYDYAPAA
(motif SEQ ID NO: 3)
(SEQ ID NO: 26)
MMSYVANLVIKSFDRASVVYVEDVVDSSRATCVENGGDDDDSGYDYAPAA
(motif SEQ ID NO: 3)
(SEQ ID NO: 27)
MAVVSHNNAEGRLYESTQTWPIAYLQIGGQENGGDDDDDDCDVAPAA
(motif SEQ ID NO: 4)
(SEQ ID NO: 63)
MVVFICKEEYGVPLSNDWAATHEFGHKFCISNEGDDDDDDNGVIDVAPAA
(motif SEQ ID NO: 5)
(SEQ ID NO: 65)
MVVFLCKEEYGVLLGNDWAATHEFGHNFCISNEGDDDDDDNGVIDVAPAA
(motif SEQ ID NO: 5)
(SEQ ID NO: 74)
MSFTSKVIALWCKKHGNDDGVDVYDAPAATACIEGNVCNWHGDFVSFVPV
ALVEGDDDDDDGGYDYAPAA
(motif SEQ ID NO: 7)
(SEQ ID NO: 75)
MSFTSKVIAPWCKKHGNDDVVDAPAATTFIGGNVCNWHGDFVSFVPIAYM
EGDDDDDGGYDYAPAA
(motif SEQ ID NO: 6)
(SEQ ID NO: 98)
MAPVSEASPLVHQDGGIIASFAVYAGAPCCSARGRMAETDGDDDDDDYDC
APAA
(motif SEQ ID NO: 8)
(SEQ ID NO: 99)
MAIAKSECERLAWALLLESNLLVGNRRSNGDDDDDDVDVAPAA
(motif SEQ ID NO: 9)

Specifically, the iron-regulated polypeptide as described herein can have one or more biological activities including induction of ferric reduction activity or activation of one or more transcriptional factors for Fe homeostasis in plants such as AtbHLH38, AtbHLH39, AtFIT or any combinations thereof. A variety of methods known in the art can be used to assess or determine such biological activities of the iron-regulated polypeptide of the present invention.

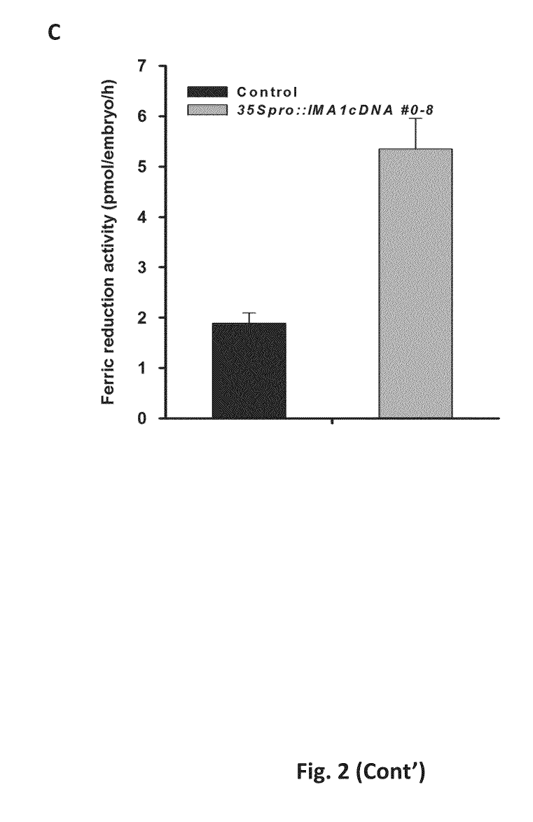

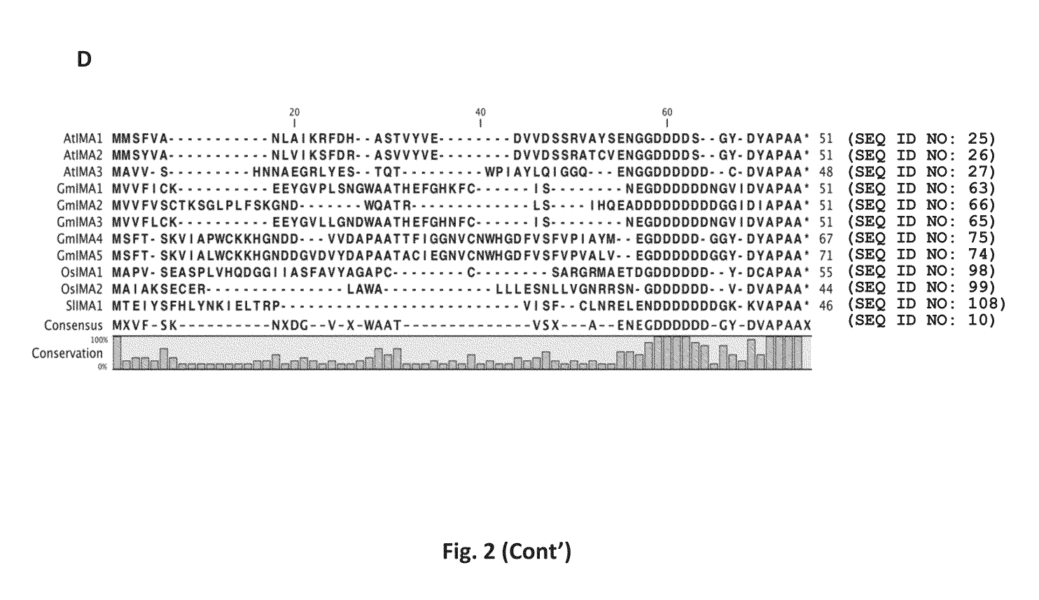

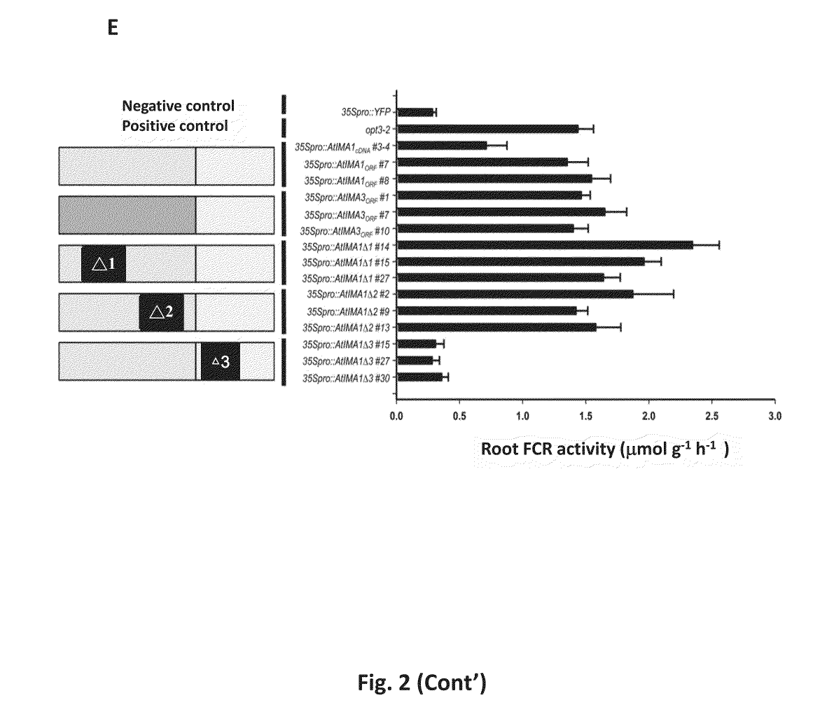

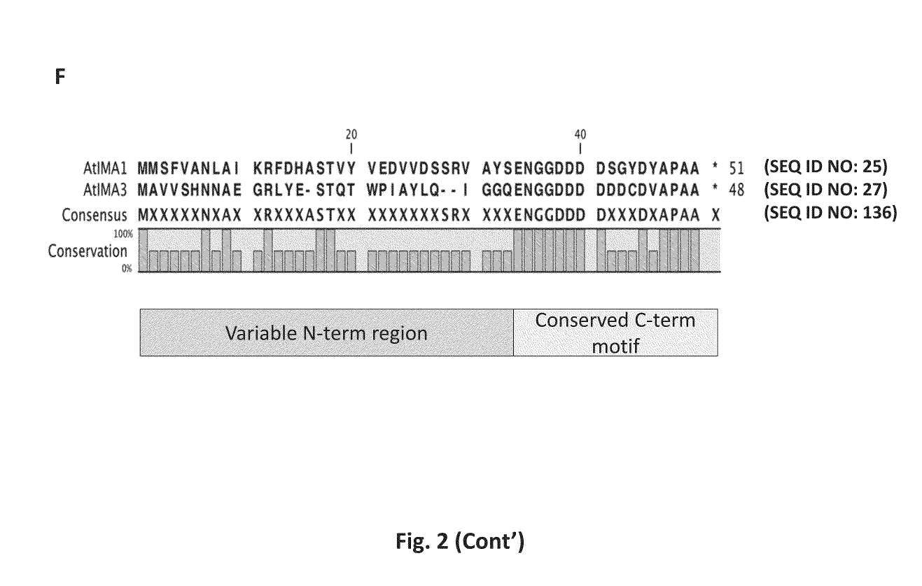

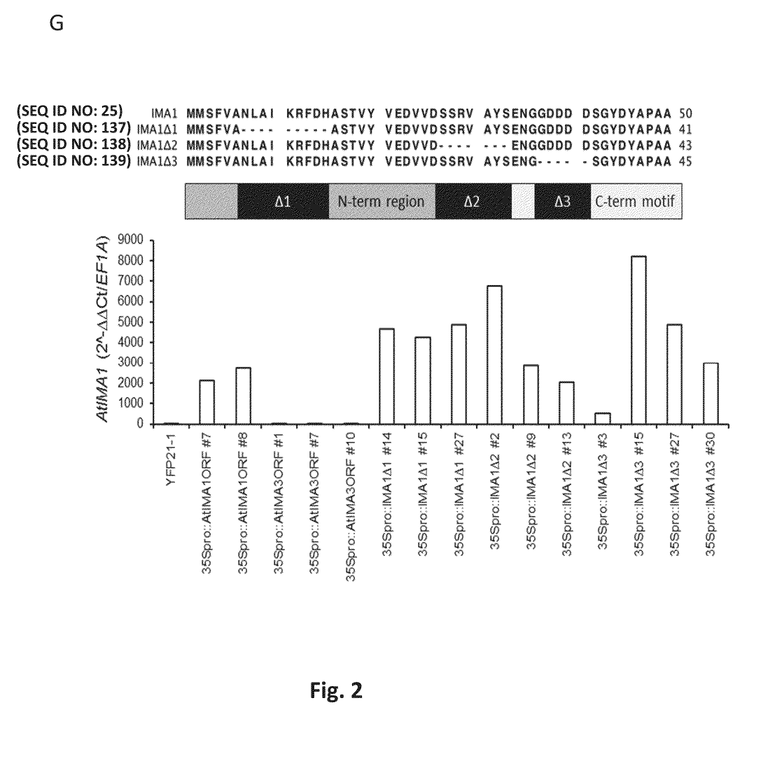

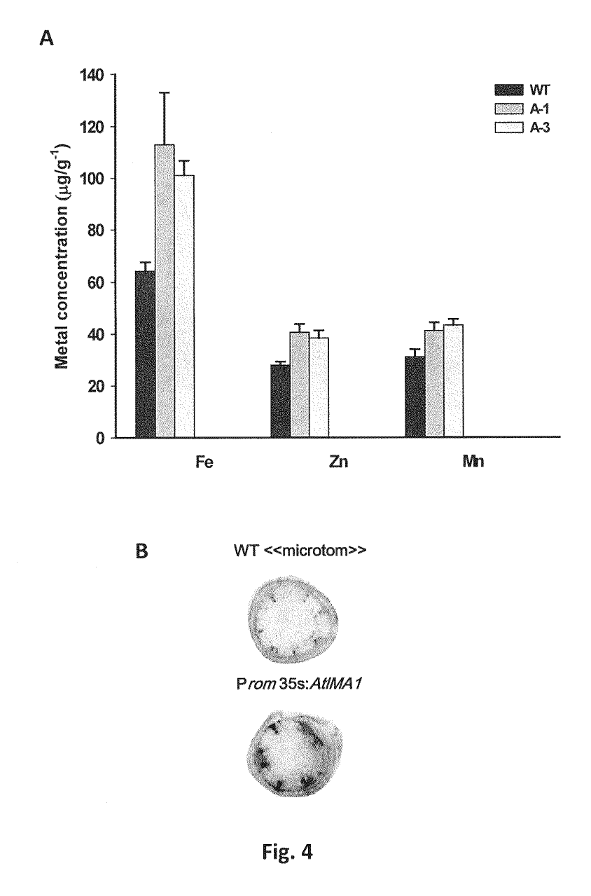

According to the present invention, a transgenic plant overexpressing an iron-regulated polypeptide as described herein can take up trace elements (Fe, Zn, Mn) from soils and accumulate these trace elements in a higher level, as compared with a control type plant (wild type, non-transgenic). As used herein a “control plant” means a plant that does not contain the recombinant DNA for expressing a protein that imparts an enhanced trait. A suitable control plant can be a non-transgenic plant of the parental line used to generate a transgenic plant, e.g. devoid of recombinant DNA. In some embodiments, the transgenic plant of the invention overexpressing an iron-regulated polypeptide as described herein exhibits an increase in Fe, Zn or Mn, which is about 1.1 fold to 15 fold of that of a control plant being grown under the same conditions. In some embodiments, the trace elements can be accumulated in aerial tissues, such as leaves or shoots, and also in seeds or fruits, or roots. As shown in examples below, the mineral nutrient analysis of the transgenic plant of the invention (transformed by 35Spro::At1g47400_{cDNA}) shows a 15-fold increase in Fe, 6.8-fold in Mn and 3.4-fold higher Zn concentrations relative to the wild type, and importantly, seed Fe concentration is increased 2- to 3-fold in transgenic lines. See FIG. 2B. In a specific example, a transgenic tomato plant carrying a recombinant construct expressing an iron-regulated polypeptide as described herein (35Spro::AtIMA1_{cDNA}) relearns a 60% increase in Fe levels in the fruit compared with a wild type tomato plant without the recombinant construct. See FIG. 4.

According to the present invention, it is also found that the conserved C-terminal motif is critical for the function of the iron-regulated polypeptide as described herein. As shown in the examples below, transgenic lines that contain either the full coding sequence of the iron-regulated polypeptide (e.g. A. thaliana IMA1, SEQ ID NO: 25) or chimeric AtIMA1 with deletions in the part encoding the non-conserved amino acids (e.g. 35Spro::IMA1_{ORF}Δ1 (SEQ ID NO: 137) and 35Spro::IMA1_{ORF}Δ2 (SEQ ID NO: 138)) exhibit a full and comparable ferric reduction activity (a prerequisite step prior to Fe uptake in plant); however, in contrast, the ferric reduction activity is almost abolished in the transgenic lines transformed with the chimeric AtIMA1 with deletions in the C-terminal motif (e.g. 35Spro::IMA1_{ORF}Δ3 (SEQ ID NO: 139)). FIG. 2E.

Plants to which the present invention can be applied include both monocotyledon and dicotyledon. Examples of monocotyledons include but are not limited to rice, barley, wheat, rye, oat, corn, bamboo, sugar cane, onion, leek and ginger. Examples of the dicotyledons include, but are not limited to Arabidopsis thaliana, eggplant, tobacco plant, red pepper, tomato, burdock, crown daisy, lettuce, balloon flower, spinach, chard, sweet potato, celery, carrot, water dropwort, parsley, Chinese cabbage, cabbage, radish, watermelon, melon, cucumber, pumpkin, gourd, strawberry, soybean, mung bean, kidney bean, and pea. Preferably, the transgenic plant of the invention is edible.

A plant tissue, plant part or plant cell of a transgenic plant of the invention is also provided. Particularly, the plant tissue, plant part or plant cell of a transgenic plant of the invention includes, for example, leaves, roots, fruits or seeds, wherein the contents of trace elements (Fe, Zn, Mn) are enhanced as compared to those from a control plant. Preferably, the plant tissue, plant part or plant cell is edible.

The present invention thus also provides a method for biofortification comprising growing a transgenic plant of the invention or its seed or other propagating materials under a condition to express the iron-regulated polypeptide, sufficient for a content of a trace element to increase in the transgenic plant, wherein the trace element is selected from the group consisting of iron (Fe), zinc (Zn) and manganese (Mn). Such transgenic plant or its plant parts or tissues (preferably edible parts), wherein the contents of trace elements (Fe, Zn, Mn) are enhanced, as compared to a control plant, are then selected and harvested.

In particular, the present invention provides a method for producing a transgenic plant with increased content of trace element(s), comprising (a) transforming a plant cell with a recombinant polynucleotide comprising a nucleotide sequence encoding an iron-regulated polypeptide, as described herein, to obtain a recombinant plant cell; and (b) growing the recombinant plant cell obtained in (a) to generate a transgenic plant. To select a pant with desired traits, the method of the invention further comprises (c) selecting a transgenic line which accumulates a trace element (Fe, Zn, Mn) in a higher level, as compared with a wild type plant (non-transgenic) while being grown under the same conditions.

In some embodiments, the transgenic plant according to the present invention or its parts are edible and thus can be eaten directly as food for use in supplementing a trace element in a subject.

In some embodiments, the transgenic plant according to the present invention or its parts are further processed such as being dried, ground or lyophilized, to form a plant product which can be then formulated to a composition, which can for example used as a nutrient supplement/formulation or a pharmaceutical composition for treating trace element deficiency. The present invention thus also provides a method of supplementing a trace element in a subject, comprising administering an effective amount of a transgenic plant, a plant product made therefrom or a composition comprising the plant product as described herein. The method of the invention can be used to treat trace element deficiency, such as deficiency of Fe, Zn or Mn or a combination thereof. For example, Fe deficiency can cause anemia. Also provided is use of a transgenic plant for manufacturing a plant product or a composition comprising the plant product for supplementing a trace element or treating trace element deficiency in a subject in need.

Specifically, a composition of the present invention, comprising a product made from the transgenic plant according to the present invention or its parts, is formulated with an acceptable carrier to facilitate delivery. “Acceptable” means that the carrier is compatible with the active ingredient in the composition, and preferably can stabilize said active ingredient and is safe to the individual receiving the treatment. Said carrier may be a diluent, vehicle, excipient, or matrix to the active ingredient. Some examples of appropriate excipients include lactose, dextrose, sucrose, sorbose, mannose, starch, Arabic gum, calcium phosphate, alginates, gum, gelatin, calcium silicate, microcrystalline cellulose, polyvinyl pyrrolidone, cellulose, sterilized water, syrup, and methylcellulose. The composition may additionally comprise lubricants, such as talc, magnesium stearate, and mineral oil; wetting agents; emulsifying and suspending agents; preservatives, such as methyl and propyl hydroxybenzoates; sweeteners; and flavoring agents. The composition of the present invention can provide the effect of rapid, continued, or delayed release of the active ingredient after administration to the patient.

The composition of the invention can be formulated in any forms as desired using conventional techniques. In a certain example, the composition of the invention is in the form of powder, more specifically are lyophilized powders, which may be further loaded into capsules. In other examples, the composition of the invention is in the form of tablets, pills, particles, lozenges, sachets, cachets, elixirs, suspensions, emulsions, solutions, syrups, soft and hard gelatin capsules, suppositories, or sterile injectable solutions. The composition may be delivered through any medically acceptable route such as orally, parentally (e.g. intramuscularly, intravenously, subcutaneously, interperitoneally), topically, transdermally, by inhalation and the like.

The term “effective amount” used herein refers to the amount of an active ingredient to confer a therapeutic effect in a treated subject. For example, an effective amount for supplementing a trace element is an amount that can provide a desired content of the trace element in a subject in need, e.g. in a condition of malnutrition.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

### Examples

Iron deficiency is severely affecting the performance and nutritional quality of plants and is the most frequent cause of anemia in humans. Co-expression and sequence motif analysis of transcriptome data from Fe-deficient rice and Arabidopsis plants identified a novel family of peptides that share a short C-terminal amino acid sequence motif conserved in numerous, highly diverse peptides across angiosperms. We named this peptide sequence IRON MAN (IMA), referring to its ability to trigger iron and manganese accumulation through activation of iron uptake genes. Deletions in the C-terminal motif of recombinant IMA peptides completely abolished this function. IMA orthologs are highly responsive to the iron status in various species independent on the strategy by which iron is acquired. IMA is critical in iron deficiency signaling in plants, acting early in the cascade that controls uptake, transport and cellular homeostasis of iron. Manipulating the expression of IMA peptides represents a novel strategy for iron bio-fortification in crops.

1. Materials and Methods

1.1 Construction of the Rice Gene Co-Expression Network

To identify Fe-responsive sequence motifs of unknown function that are conserved between rice and Arabidopsis, oligonucleotide sequences of the Affymetrix GeneChip rice genome microarray probes were mapped using the BLASTN program (e-value <9.9e-6) against the transcripts from the V7 release of the Rice Pseudomolecules and Genome Annotation database, and a co-expression network of Fe-responsive rice genes was constructed using a database of 2,700 publicly available microarray hybridizations retrieved from ArrayExpress (www.ebi.ac.uk/arrayexpress/). The 1,349 probes that showed >5-fold signal change in response to Fe-deficiency in the microarray experiments conducted by Zheng et al. (15) were used as input to compute a co-expression network with a Pearson correlation coefficient cutoff P >0.6 using the MACCU software for the pair wise correlation of gene expression (24). In order to restrict the network to processes closely related to Fe homeostasis, Fe-responsive genes listed in (1) and their rice orthologs, as well as all transporters from the ZIP, YSL and NRAMP families that were present in the network were selected to generate a new network consisting of these genes and their first neighbors. Arabidopsis orthologs were assigned to rice loci using the InParanoid software. When no ortholog was found, the closest Arabidopsis sequelog was assigned to the rice locus. In cases of ambiguous assignment, we used a conservative approach and matched a single rice locus to several Arabidopsis genes.

1.2 Amino Acid Sequence Motif Analysis

Sequences of candidate proteins with unknown functions were retrieved from various databases. These sequences were used as an input for the MEME suite 4.9.1 online tool (24), together with Arabidopsis proteins of unknown function from the gene network published in Rodríguez-Celma et al. (16). Motif discovery was performed with the Multiple Em for Motif Elicitation tool and the discovered motifs were then searched in the input sequences using the Motif Alignment and Search Tool (MAST). The IMA motif was the only highly significant motif resulting from this analysis. We identified genes encoding peptides containing similar motifs in C-terminal position in transcriptomes of Fe-deficient tomato (19), rice (15) and soybean (20), and used all these sequences to refine the consensus sequence of the motif.

1.3 Sequence Alignment

We retrieved about 130 individual sequences of proteins harboring the IMA motif in C-termal position from Uniprot, NCBI, individual genome annotation project websites, and EST databases. The alignment was performed using the CLC sequence viewer software. The alignment was manually adjusted and used to generate a neighbor-joining tree.

1.4 Gene Expression Analysis

Arabidopsis (Arabidopsis thaliana (L.) Heynh., Col-0) plants were grown in a growth chamber on media described previously (25). RNA was extracted using the RNeasy Kit (Qiagen) and cDNA was synthesized using SuperScript III reverse transcriptase (Life Technologies). Real-time RT-PCR was carried out in an ABI Prism 7500 Sequence Detection System (Applied Biosystems). All quantitative RT-PCR runs were performed and analyzed as detailed previously (22). Primers used for qRT-PCR are listed in Table 1.

TABLE 1
Oligonucleotides used for qRT-PCR

| | | | SEQ |
|---|---|---|---|
| Name | Target gene | Sequence (5′-3′) | ID NO |
| qFER1 F | At5g01600 | TCCCCAGTTAGCTGATTTCG | 140 |
| qFER1 R | AtFER1 | CTTTGCCGATCATCCTTAGC | 141 |
| qAtIRT1F | At4g19690 | CGTGCGTCAACAAAGCTAAA | 142 |
| qAtIRT1R | AtIRT1 | TCTGGTTGGAGGAACGAAAC | 143 |
| qFRO2f | Ag1g01580 | GATCGAAAAAAGCAATAACGGTGGTT | 144 |
| qFRO2r | AtFRO2 | GATGTGGCAACCACTTGGTTCGATA | 145 |
| qFITf | At2g28160 | CAGTCACAAGCGAAGAAACTCA | 146 |
| qFITr | AtFIT1 | CTTGTAAAGAGATGGAGCAACACC | 147 |
| NIG1 QS | At1g47400 | ATGTCTTTTGTCGCAAACTTGGC | 148 |
| NIG1 QA | ΛIRP1/ΛtIMΛ1 | CACCACCATTCTCACTATATGCCAC | 149 |
| qAtbHLH38f | At3g56970 | GACGGTACCACAGACTTATGAAGT | 150 |
| qAtbHLH38r | AtBHLH38 | TAAGCTCTTTGAAACCGTTTCAGGA | 151 |
| qAtbHLH39f | At3g56980 | GACTTATGGAGCTGTTACAGCGGT | 152 |
| qAtbHLH39r | ΛtBHLH39 | CTTCAAGCTTCGAGAAACCGTCGCA | 153 |
| q47400F | At1g47400 | TGATTGTAATTTAGGAGGAAACAAAA | 154 |
| q47400R | AtIMA1 | TCAATCCACAAGTAAACATCTATGG | 155 |
| qAtEFf | At5g19510 | GCTGTTCGTGGTGTTGAGATGC | 156 |
| qAtEFr | AtEF1B | AGGCTCTGAGGTGAGGAAGTCT | 157 |
| q37066F | At2g30766 | TGAGTCACAACAACGCAGAAGG | 158 |
| q37066R | AtIMA3 | GGCCAAGTCTGAGTTGATTCGT | 159 |
| qIMA1alldelF | At1g47400 | TGAGTCACAACAACGCAGAAGG | 160 |
| qIMA1alldelR | AtIMA1, Δ1, Δ2, Δ3 | GGCCAAGTCTGAGTTGATTCGT | 161 |
| EF1α-QS | AT5G60390 | GAGCCCAAGTTTTTGAAGA | 162 |
| EF1α-QA | AtEF1A | CTAACAGCGAAACGTCCCA | 163 |

1.5. Generation of Transgenic Lines

Full length AtIMA1 cDNA was amplified with engineered BamHI sites and cloned into BamHI digested and de-phosphorylated pBIN-pROK2 to generate the pROKIMA1 binary vector, which was used for Arabidopsis (lines 35Spro::IMA1_{cDNA} 0-8, 1-4, 2-1 and 3-4) and tomato transformation (lines 35Spro::IMA1_{cDNA} A-1 and A-3). Constructs used for overexpression of AtIMA1 (lines 35Spro::IMA1_{oRF} #7 and #8), IMA1Δ1, IMA1Δ2, IMA1Δ3 and IMA3, the 153 bp and 144 bp open reading frames of both genes were cloned into PCR8/GW/TOPO with engineered XbaI site in 5′ and a SacI site in 3′ and obtained the plasmids pIMA1TOPO and pIMA3TOPO that were subsequently transferred into the pH2GW7 vector (26) by Gateway™ recombination, yielding the pHIMA1 and pHIMA3 vectors. IMA1 deletions were generated by PCR using pIMA1TOPO as a template. The fragment in 5′ of the deletion site was amplified using the M13 forward primer, and a phosphorylated reverse primer complementary to the sequence adjacent to the deletion site. The fragment in 3′ of the deletion site was amplified using a forward phosphorylated primer complementary to the sequence adjacent to the site and the M13 reverse primer. The two amplicons were digested with XbaI or SacI, respectively, and ligated together into the pIMA1TOPO vector from which the IMA1 full-length CDS had been removed by XbaI-SacI digestion. Plasmids pIMAΔ1TOPO, pIMAΔ2TOPO and pIMAΔ3TOPO were obtained this way and recombined with pH2GW7 in order to produce the binary pHIMA1Δ1, pHIMA1Δ2 and pHIMA1Δ3. The artificial microRNA targeting both IMA1 and IMA2 was generated according to Schwab et al. (27) using the online Web MicroRNA Designer tool. The pHamiR-IMA1 vector was produced by engineering a miR319a backbone by site-directed mutagenesis in order to target the TTACTAATAGGAGACAATCAT sequence (SEQ ID NO: 185) common to both genes. The chimeric amiR-IMA1 gene was cloned into the pENTR™/D/TOPO vector and subsequently inserted into pH2GW7 using the gateway system. Agrobacterium tumefaciens strain GV3101 (pMP90) was used to transform Arabidopsis Col-0 plants via the floral dip method (28); strain LBA4404 was used to transform tomato MicroTom. Primers used for cloning are listed in Table 2.

TABLE 2
Oligonucleotides used for cloning

| | | | SEQ |
|---|---|---|---|
| Name | Target gene | Sequence (5′-3′) | ID NO |
| IMA1_{cDNA}-BamHI F | At1g47400 | GCGGGATCCCATCAACATTTGAAGCTCA | 164 |
| | IMA1 cDNA | | |
| IMA1_{cDNA}-BamHI R | | CGCGGATCCGGAAACTAGCAATATTATAA | 165 |
| IMA1_{CDS}-XbaI F | At1g47400 | AAATCTAGAATGATGTCTTTTGTCGCAAACTTG | 166 |
| | IMA1 CDS | | |
| IMA1_{CDS}-SacI R | | AAAGAGCTCTCACGCAGCAGGAGCATAATC | 167 |
| IMA3_{CDS}-XbaI F | At2g30766 | AAATCTAGAATGGCAGTGGTGAGTCACAACAACGC | 168 |
| | IMA3 CDS | | |
| IMA3_{CDS}-SacI R | | AAAGAGCTCTCAAGCCGCCGGTGCAACG | 169 |
| IMA1Δ1 F | At1g47400 | PO₃²⁻-GCTTCCACCGTGTATGTT | 170 |
| IMA1Δ1 R | IMA1Δ1 CDS | PO₃²⁻-TGCGACAAAAGACATCAT | 171 |
| IMA1Δ2 F | At1g47400 | PO₃²⁻-GAGAATGGTGGTGATGACGATG | 172 |
| IMA1Δ2 R | IMA1Δ2 CDS | PO₃²⁻-ATCTACCACATCTTCAACATACACGG | 173 |
| IMA1Δ3 F | At1g47400 | PO₃²⁻-AGTGGCTATGATTATGCTCCTGC | 174 |
| IMA1Δ3 R | IMA1Δ3 CDS | PO₃²⁻-ACCATTCTCACTATATGCCACTCGAGAAC | 175 |
| M13 F | pIMA1TOPO | GTTGTAAAACGACGGCCAGTC | 176 |
| M13 R | pIMA1TOPO | TGCCAGGAAACAGCTATGACC | 177 |
| I-amiIMA1 | miR319a | GATTACTAATAGGAGACAATCATTCTCTCTTTTGTATTCC | 178 |
| II-amiIMA1 | backbone in | GAATGATTGTCTCCTATTAGTAATCAAAGAGAATCAATGA | 179 |
| | pRS300 | | |
| III-amiIMA1 | | GAATAATTGTCTCCTTTTAGTATTCACAGGTCGTGATATG | 180 |
| IV-amiIMA1 | | GAATACTAAAAGGAGACAATTATTCTACATATATATTCCT | 181 |
| primer A | miR-IMA1 | CTGCAAGGCGATTAAGTTGGGTAAC | 182 |
| primer B | | GCGGATAACAATTTCACACAGGAAACAG | 183 |

1.6 Ferric Reductase Activity

Ferric reductase activity was measured as described in Grillet et al. (17) using sets of roots from five to ten seedlings (10-25 mg FW) incubated for 1 h in the dark with mild shaking, in 2 mL assay solution consisting of 100 μM Fe^{III}-EDTA, 300 μM bathophenanthroline disulfonate (BPDS) in 10 mM 2-(N-morpholino)ethanesulfonic acid (MES) at pH 5.5. Fe^{II}-BPDS₃ concentration was determined after reading the absorbance at 535 nm on a PowerWave XS2 plate reader (BioTek Instruments, USA).

1.7 Microarray Experiments

The Affymetrix GeneChip Arabidopsis ATH1 Genome Array was used for microarray analysis. Data files were imported into GeneSpring GX11 (Agilent) by applying robust multiarray average (RMA) for per chip normalization. The data were then filtered on expression above 100 in the raw data. A two-way ANOVA statistical analysis was applied to determine differentially expressed genes, and a P value of <0.05 was considered significant. Genes that were either up-regulated or down-regulated more than 1.5-fold were selected.

1.8 Determination of Mineral Concentrations

Roots and shoots from 3-week-old wild-type and 35Spro::AtIMA1_{cDNA} plants grown under control conditions were harvested separately. Mineral nutrient analysis was determined by inductively coupled plasma optical emission spectrometry (ICP-OES). Five plants were harvested per treatment and genotype, dried in a conventional oven at 60° C. and ground in a stainless steel mill. Aliquots (˜0.15 g dry weight) were placed in 100 mL borosilicate glass tubes, 3 mL of ultra-pure nitric acid was added, and the material was pre-digested overnight at room temperature. Subsequently, the tubes were placed in a digestion block (Magnum Series, Martin Machine, Ivesdale, Ill., USA) and maintained at 125° C. for a minimum of 4 h (with refluxing). The tubes were then removed from the block, cooled for 5 min, 2 mL of hydrogen peroxide were added, and the samples were returned to the block for 1 h at 125° C. This hydrogen peroxide treatment was repeated twice. Finally, the digestion block temperature was raised to 200° C., and samples were maintained at this temperature until dry. Once cooled, samples were resuspended in 15 mL 2% ultra-pure nitric acid (w/w) overnight, then vortexed and transferred to plastic storage tubes until analysis. Elemental analysis was performed using ICP-OES (CIROS ICP Model FCE12; Spectro, Kleve, Germany). The instrument was calibrated daily with certified standards. Tomato leaf standards (SRM 1573A; National Institute of Standards and Technology, Gaithersburg, Md., USA) were digested and analyzed along with the Arabidopsis samples to ensure accuracy of the instrument calibration.

1.9 Perls' Staining for Fe(III)

Arabidopsis seedlings were vaccum infiltrated with Perls' solution (2% HCl and 2% Potassium ferrocyanide) for 15 minutes and incubated for another 30 minutes. Samples were then rinsed three times with distilled water. For Fe localization in embryos, the Perls' staining was intensified with diaminobenzidine (DAB) as described in Roschzttartdz et al. (29). Briefly, embryos were incubated for 1 h in a methanol solution containing 0.01 M sodium azide and 0.3% H₂O₂, and washed with 100 mM sodium phosphate buffer pH 7.4. Staining was then intensified by 10 min incubation in 0.025% DAB, 0.005% H₂O₂ and 0.005% CoCl₂).

2. Results

2.1 Identification of the G-D-D-D-D-Spacer-D-x-A-P-A-A Sequence Motif

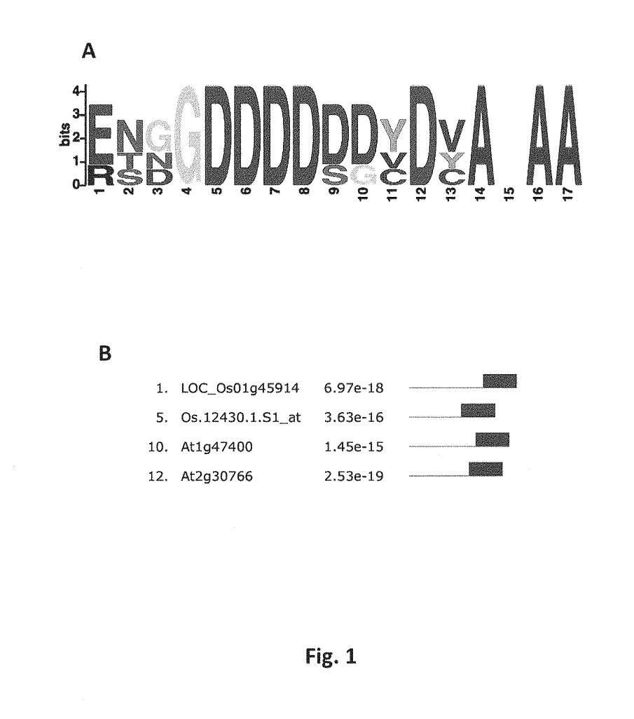

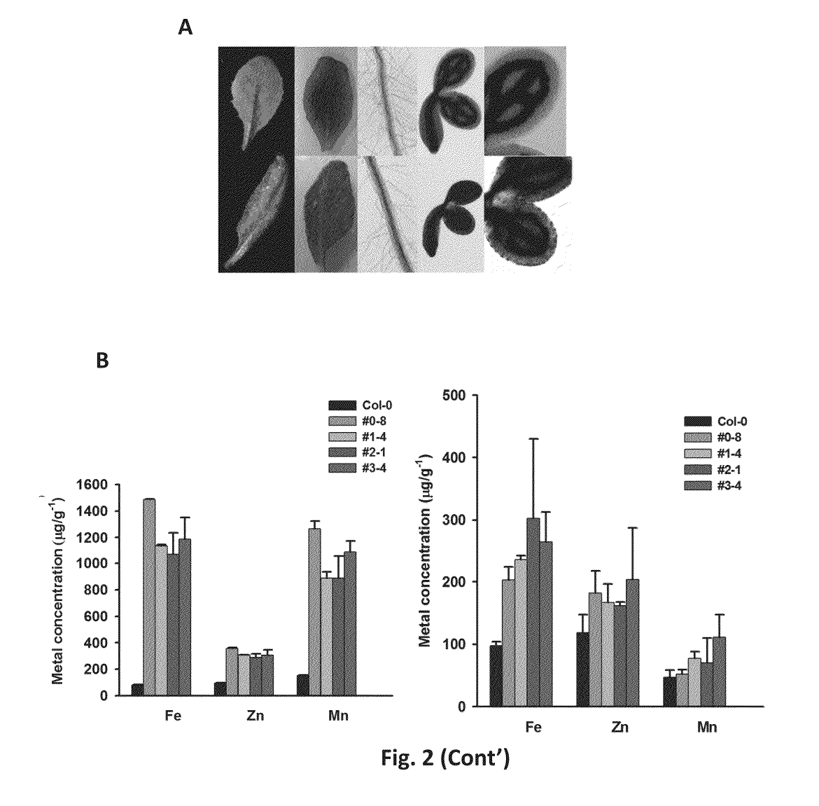

Similarities in the proteins controlling Fe sensing and acquisition between rice and Arabidopsis suggest Fe signaling nodes that are conserved across species. To discover such nodes, we aimed at identifying sequence motifs in Fe-responsive proteins of unknown function in two model species with well-explored Fe deficiency responses, rice and Arabidopsis. To this end, we constructed a co-expression network comprised of Fe-responsive rice genes that showed signal changes greater than 5-fold in response to Fe deficiency (15) using a database of 2,700 publicly available microarray hybridizations. In order to restrict the network to processes closely related to Fe homeostasis, we generated a sub-network consisting of the rice orthologs of Fe homeostasis genes listed in Kobayashi et al. (1), all transporters from the ZIP, YSL and NRAMP families, and the nodes that were connected to at least two of these genes at the first degree. We then assigned Arabidopsis orthologs or the closest sequelogs to the nodes in the network. Sequences of 14 unknown rice proteins in this network and Fe-responsive Arabidopsis genes encoding proteins of unknown function identified in a previously conducted RNA-seq survey (At1g47400, At2g14247, At1g13609, At2g30760 and At2g30766; 16) were then screened for conserved sequence motifs. A C-terminal amino acid sequence, G-D-D-D-D-spacer-D-x-A-P-A-A (FIG. 1A), was found to be conserved in two Arabidopsis (At1g47400 and At2g30766) and two rice proteins, corresponding to LOC_Os01g45914 (probe sets Os.12629.1.S1_at and Os.12629.1.S2_at) and to a non-annotated transcript encoded by a gene located between LOC_Os07g04910 and LOC_Os07g04930 that we designated as LOC_Os07g04920 (probe sets Os.12430.1.S1_at and Os.48053.1.A1_at) (FIG. 1B).

2.2 the G-D-D-D-D-Spacer-D-x-A-P-A-A Motif is Critical for the Function of IMA Peptides.

Transgenic plants ectopically expressing At1g47400 under the control of the CaMV 35S promoter (35Spro::At1g47400_{cDNA}) displayed necrotic spots in the leaves, resembling Fe toxicity symptoms (FIG. 2A). Plants overexpressing At2g30766 showed a similar phenotype. Perls' staining confirmed that these necrotic spots were caused by excess Fe accumulation (FIG. 2A). High Fe levels were also observed in the stele (FIG. 2A). Mineral nutrient analysis of 35Spro::At1g47400_{cDNA} plants by ICP-OES confirmed dramatically increased levels of Fe, zinc (Zn) and manganese (Mn) (FIG. 2B). Aerial tissues showed a 15-fold increase in Fe, 6.8-fold in Mn and 3.4-fold higher Zn concentrations relative to the wild type. Importantly, seed Fe concentration was increased 2- to 3-fold in transgenic lines (FIG. 2B). Notably, the ferric reduction activity of embryos, a prerequisite step prior to Fe uptake (17), was significantly increased in 35Spro::At1g47400_{cDNA} plants when compared to the wild type (FIG. 2C).

To classify peptides containing the G-D-D-D-D-spacer-D-x-A-P-A-A sequence motif, we named the encoding genes IRON MAN (IMA), referring to the over-accumulation of iron, zinc and manganese that is caused by their ectopic expression. The Arabidopsis genome harbors six IMA genes that are all responsive to the Fe regime. AtIMA1 (At1g47400), AtIMA2 (At1g47395) and AtIMA3 (At2g30766) are highly expressed in both leaves and roots of Fe-deficient plants (16,18). By contrast, AtIMA4-6 that we assigned as At1g47401 (AtIMA4), At1g47406 (AtIMA5) and At1g47407 (AtIMA6) are lowly expressed and are not included in the TAIR10 genome annotation.

Putative IMA orthologs are among the most strongly Fe-responsive genes in roots and leaves of species for which data on Fe deficiency-induced changes in transcriptional profiles are available; i.e. tomato (Probe ID TC209134_{—}260_{—}40_S, designated SlIMA1; 19), rice roots/leaves (Os01g45914; designated OsIMA1; 15), rice leaves (transcript ID gi:297606717, designated OsIMA2; 15) and soybean (Glyma02g45170/GmIMA1, Glyma18g14490/GmIMA2, Glyma14g03580/GmIMA3, Glyma17g12804/GmIMA4, Glyma05g08181/GmIMA5; 20). Induction of OsIMA1 and OsIMA2 by Fe deficiency was much more pronounced in leaves when compared to roots (525- vs 39-fold for OsIMA1; OsIMA2 was induced 2,252-fold in leaves only (15). Amino acid alignments of the encoded peptides show high sequence variability except for the conserved IMA sequence (FIG. 2D).

AtIMA1 and AtIMA3 share only 38% sequence identity (FIG. 2F), which is mainly confined to the C-terminus motif (FIG. 2D). Decreasing the expression of AtIMA1 and AtIMA2 using an artificial microRNA construct did not impair the ability of the plants to induce their root FCR activity when subjected to Fe deficiency. These data suggest that IMA genes in Arabidopsis are functionally redundant and that the conserved C terminus in IMA peptides is critical for their function. To test this assumption, we generated transgenic lines that contain either the full coding sequence of AtIMA1 (35Spro::IMA1_{ORF}) or chimeric AtIMA1 with deletions either in the part encoding the non-conserved amino acids (35Spro::IMA1_{ORF}Δ1 (SEQ ID NO: 137) and 35Spro::IMA1_{ORF}Δ2 (SEQ ID NO: 138)) or in the C-terminal motif (35Spro::IMA1_{ORF}Δ3 (SEQ ID NO: 139)) (FIG. 2E; FIG. 2G). Inferred from their ability to induce the root ferric-chelate reductase activity, IMA1Δ1 and IMA1Δ2 proteins were fully functional whereas partial deletion of the conserved motif completely abolished this property (FIG. 2E). This finding demonstrates that the conserved C-terminal motif of IMA is critical for its function.

Peptides harboring the IMA motif are present in the genomes of all angiosperms including the anciently diverged species Amborella trichopoda, suggesting that IMA is conserved in the flowering plant lineage. Based on the available genomic data, we identified 125 genes encoding putative IMA sequences in 29 plant species. See Table 3. We failed to detect IMA-encoding sequences in the genomes of gymnosperms, ferns, algae or fungi, indicating that IMA has emerged at an early stage of angiosperm evolution. All IMA genes are either annotated as encoding unknown proteins or are not annotated at all in the respective genomes.

TABLE 3
125 genes encoding putative IMA sequences in 29 plant species

| | SEQ | | | |
|---|---|---|---|---|
| Gene name | ID NO | Organism | Locus | |
| Ae. tauschii | 11 | Aegilops | | MAPASKVMSCHIVQDGGIADYAVYAAAPCDAWCGGRHRKA |
| IMA1 | | tauschii | | ESD [symbol] DYY [symbol] |
| Am. trichopoda | 12 | Amborella | | MYQRYDAPFVGQKWHQKRIGE [symbol] DDDY [symbol] |
| IMA1 | | trichopoda | | |
| Am. trichopoda | 13 | Amborella | | MLQRYDAPFVGQKWHQKRIGE [symbol] DDDY [symbol] |
| IMA2 | | trichopoda | | |
| Am. trichopoda | 14 | Amborella | | MLDRHDAHLGCQKWHQKKILRTEGDDDDDDDDYDCAPAT |
| IMA3 | | trichopoda | | |
| Am. trichopoda | 15 | Amborella | | MLDRHDAHLCCLKWHQKKILRTE [symbol] DDDDY [symbol] |
| IMA4 | | trichopoda | | |
| Am. trichopoda | 16 | Amborella | | MASEENPPNRRDDDDDDDDYDCAPAT |
| IMA5 | | trichopoda | | |
| Am. trichopoda | 17 | Amborella | | MASEENRE [symbol] DDDY [symbol] |
| IMA6 | | trichopoda | | |
| A. lyrata | 18 | Arabidopsis | | MMSFVANLAIKSLDRASAVYVEDVVDSSRVAYGENG [symbol] |
| IMA1 | | lyrata | | [symbol] SGY [symbol] |
| A. lyrata | 19 | Arabidopsis | | MMSFVANLVIKSFYRASAMYVEDMVDSSRATCLENG [symbol] |
| IMA2 | | lyrata | | [symbol] SGY [symbol] [symbol] |
| A. lyrata | 20 | Arabidopsis | | MMYFFANLVSKSFDRASAVYVEDVVDCSRATCVENG [symbol] |
| IMA3 | | lyrata | | [symbol] SGY [symbol] [symbol] |
| A. lyrata | 21 | Arabidopsis | | MISVTEFILCIDDNVGGTCIGGEVVISGQAFVYAQSVYV |
| IMA4 | | lyrata | | EDGDNDDDDIYDIAPAA |
| A. lyrata | 22 | Arabidopsis | | MISVSEIVLYIHENVYETSIGVNIANNDKVFEYAQATFV |
| IMA5 | | lyrata | | ENGDNDDDVIYDYAPAA |
| A. lyrata | 23 | Arabidopsis | | MSSLSEFVLSIYDHVSESCVGSDTTSYDQEIKSRQAAYA |
| IMA6 | | lyrata | | ENGDQDDDDIYDYAPAA |
| A. lyrata | 24 | Arabidopsis | | MVSIYKFVLCKCDQVRETCIRGDVTYNNGEFEYHQVAFI |
| IMA7 | | lyrata | | EN [symbol] DIIY [symbol] [symbol] |
| A. thaliana | 25 | Arabidopsis | At1g474 | MMSFVANLAIKRFDHASTVYVEDVVDSSRVAYSENG [symbol] |
| IMA1 | | thaliana | 00 | [symbol] SGY [symbol] [symbol] |
| | | | | (C-motif: SEQ ID NO: 3) |
| A. thaliana | 26 | Arabidopsis | At1g473 | MMSYVANLVIKSFDRASVVYVEDVVDSSRATCVENG [symbol] |
| IMA2 | | thaliana | 95 | [symbol] SGY [symbol] Y [symbol] |
| A. thaliana | 27 | Arabidopsis | At2g307 | MAVVSHNNAEGRLYESTQTWPIAYLQIGGQENG [symbol] |
| IMA3 | | thaliana | 66 | [symbol] DDC [symbol] V [symbol] |
| A. thaliana | 28 | Arabidopsis | At1g474 | MISVSEFVLCIDDNVSGTCMRGKVVISDQAFVYAQSVYV |
| IMA4 | | thaliana | 01 | EDGDNDDDDIYDYAPAA |
| A. thaliana | 29 | Arabidopsis | At1g474 | MFSIYKFVLCKWDQVGETFIRGDVTYNNGEFEYPQVAYV |
| IMA5 | | thaliana | 06 | EN [symbol] DIIX [symbol] Y [symbol] |
| A. thaliana | 30 | Arabidopsis | At1g474 | MVSVSELVLYVHENVYETCIGVNIANNDQVFEYAQTAFV |
| IMA6 | | thaliana | 07 | ENGDNDDDVIYDYAPAA |
| A. alpina | 31 | Arabis | | MISVTEFVLCIHENVYDKCNGDGIVNNNGASDSATVENG |
| IMA1 | | alpina | | DNDDDDIYDYAPAA |
| A. alpina | 32 | Arabis | | MISVTEFVLCIDDIVYEKCIAVSGAKSIQASEFTSVENG |
| IMA2 | | alpina | | DNDDDVIYDYAPAA |
| A. alpina | 33 | Arabis | | MAVMSHNKAESRLHESTQACPSPYSVTRAHENG [symbol] |
| IMA4 | | alpina | | [symbol] DDC [symbol] V [symbol] |
| A. alpina | 34 | Arabis | | MVFVFHYVLCKYDEVCETFIEGNAIKNCAELEYSQAGYV |
| IMA3 | | alpina | | EN [symbol] DNVY [symbol] Y [symbol] |
| B. rapa | 35 | Brassica | | MYLVAHLVIKSFDGDYAVSAEDVVDTSRAAYIENG [symbol] |
| IMA1 | | rapa | | [symbol] GGY [symbol] Y [symbol] |
| B. rapa | 36 | Brassica | | MAVMSHNKAEGRLYESTQTRLVPYIQTLGQESG [symbol] |
| IMA2 | | rapa | | [symbol] DDS [symbol] V [symbol] |
| B. rapa | 37 | Brassica | | MAVMSHDKAEDRLYESAHTRPIPYNSQIVGQESG [symbol] |
| IMA3 | | rapa | | [symbol] DDS [symbol] V [symbol] |
| B. rapa | 38 | Brassica | | MFSVSEFLFCTYDNVYGGDITNNDEAVQYAQAVFSEN [symbol] |
| IMA4 | | rapa | | [symbol] DVIY [symbol] Y [symbol] |
| B. napus | 39 | Brassica | BnaA05 | MSFAANLVIINFYCASAVCVEELLDNSLGSYTENG [symbol] |
| IMA1 | | napus | g17690 | [symbol] SGY [symbol] Y [symbol] |
| | | | D | |
| B. napus | 40 | Brassica | BnaC08 | MISVREFVFCASNNNICEMCSGGVMANNDKRFEYAQAAY |
| IMA2 | | napus | g04490 | VENGDNDDDVIYDYAPAA |
| | | | D | |
| B. napus | 41 | Brassica | BnaA10 | MFSVSEFLFCTYDNVYGGDITNNDEAIQYAQAVFSEN [symbol] |
| IMA3 | | napus | g05160 | [symbol] DVIY [symbol] Y [symbol] |
| | | | D | |
| B. napus | 42 | Brassica | BnaC04 | MAVMSYNKAEGRLYESTQTRPVPYIQTVGQESG [symbol] |
| IMA4 | | napus | g41400 | [symbol] DDS [symbol] V [symbol] |
| | | | D | |
| C. sativa | 43 | Camelina | | MMTFVANLLSKSLDRASSVYVEDVVDSSRVAYGENG [symbol] |
| IMA1 | | sativa | | [symbol] SGY [symbol] Y [symbol] |
| C. sativa | 44 | Camelina | | MMTFVANLLSKSLDRASSAYVEDVVDSSRVAYGENG [symbol] |
| IMA2 | | sativa | | [symbol] SGY [symbol] Y [symbol] |
| C. sativa | 45 | Camelina | | MMTLLSKSLDRASSVYVEDVVDSSRVAYGENG [symbol] |
| IMA3 | | sativa | | SGY [symbol] Y [symbol] |
| C. sativa | 46 | Camelina | | MSFVANLVIKSFDRASTVCVEDVVDSFRAAYVENG [symbol] |
| IMA4 | | sativa | | [symbol] SGY [symbol] Y [symbol] |
| C. sativa | 47 | Camelina | | MSFVANLVIKSFDRASTVCVEDVVDSFRVAYVENG [symbol] |
| IMA6 | | sativa | | [symbol] SGY [symbol] Y [symbol] |
| C. sativa | 48 | Camelina | | MSFVANLVIKSFDRASTVCVEDVVDSFRVAYVENGGDDD |
| IMA7 | | sativa | | DSGYDYAPVA |
| C. sativa | 49 | Camelina | | MMSSVANLVIKSFDYASTVCVEDVVDSSRAAYVENG [symbol] |
| IMA5 | | sativa | | [symbol] DSGY [symbol] Y [symbol] |
| C. sativa | 50 | Camelina | | MMSSVANLVIKSFDYASTVCLEDVVDSSRAAYVENG [symbol] |
| IMA8 | | sativa | | [symbol] SGY [symbol] Y [symbol] |
| C. sativa | 51 | Camelina | | MSSVADLVIKSFNHASTVCDEDVVDTFRAAYVESG [symbol] |
| IMA9 | | sativa | | [symbol] SGY [symbol] Y [symbol] |
| C. sativa | 52 | Camelina | | MSSVADLVIKSFNHASTACDEDVVDSFRAAYVENG [symbol] |
| IMA10 | | sativa | | [symbol] SGY [symbol] Y [symbol] |
| C. sativa | 53 | Camelina | | MMPSVANLVIKSFEYVSTVCLEDVKDSSRVAYVENG [symbol] |
| IMA11 | | sativa | | [symbol] SGY [symbol] Y [symbol] |
| C. sativa | 54 | Camelina | | MAVLIVSRNNNGEGRLYESTRTQPIPYLQNGGQENG [symbol] |
| IMA12 | | sativa | | [symbol] DDC [symbol] V [symbol] |
| C. arietinum | 55 | Cicer | | MASISMIIAPRCEKHAYGEGDRFCYISTACVELEDYHSG |
| IMA1 | | arietinum | | GGDFVSPQVTYNE [symbol] DGGY [symbol] Y [symbol] |
| C. clementina | 56 | Citrus | | MAPMSSSLEGITHGNVHHRDDDSIHVYGCPYYYRNEPFE |
| IMA1 | | clementina | | GDGDDDDDDDDGCDLAPAASMEGD [symbol] DDGGY [symbol] Y |
| | | | | [symbol] |
| C. clementina | 57 | Citrus | | MSLVSKSVMPSSSWTWCKKHGDGDDDDDDGYDYAPAACI |
| IMA2 | | clementina | | EGYGDDDDDDGDYDYAPAASMEGDDDGSYDYAPAA |
| C. clementina | 58 | Citrus | | MSSSLEGITHGNVHHRDDDSIHVYGCPYYYRNEPFEGDG |
| IMA3 | | clementina | | DDDDDDDDGCDLAPAASMEGD [symbol] DDGGY [symbol] [symbol] |
| C. melo | 59 | Cucumis | | MDVSIFEPVASTMIKNIIAYKDVKCGRQFSTNLTTTIIR |
| IMA1 | | melo | | RE [symbol] DDGCY [symbol] Y [symbol] |
| C. sativus | 60 | Cucumis | | MAPISRLPCVLGLKNLGGDGGHGYREGCDCGYTTLVSMA |
| IMA1 | | sativua | | EGDSDDDDGGYDFAPAA |
| C. sativus | 61 | Cucumis | | MVSTSKSVASMMIKNNVCEDVKCSRSFPIDLTKTIIRRE |
| IMA2 | | sativua | | [symbol] DDGCY [symbol] Y [symbol] |
| E. guttata | 62 | Erythranthe | | PNHTHSNSWNLCSKRSELTSEEIRVSPNFGILITQLHDR |
| IMA1 | | guttata | | EGDDDDDDDDGGTFVAPAA |
| G. max | 63 | Glycine | | MVVFICKEEYGVPLSNDWAATHEFGHKFCISNE [symbol] |
| IMA1 | | max | | [symbol] DDNGVI [symbol] V [symbol] |
| | | | | (C-motif: SEQ ID NO: 5) |
| G. max | 64 | Glycine | | MVVFICKEEYGVPLSNGWAATHEFGHKFCISNE [symbol] |
| IMA11 | | max | | [symbol] DDNGVI [symbol] V [symbol] |
| G. max | 65 | Glycine | | MVVFLCKEEYGVLLGNDWAATHEFGHNFCISNE [symbol] |
| IMA3 | | max | | [symbol] DDNGVI [symbol] V [symbol] |
| | | | | (C-motif: SEQ ID NO: 5) |
| G. max | 66 | Glycine | | MVVFVSCTKSGLPLFSKGNDWPATRFPIHQETDDDDDDD |
| IMA2 | | max | | DDDGGIDIAPAA |
| G. max | 67 | Glycine | | MVVFVSCTKSGLPLFSKGNDWQATRLSIHQEADDDDDDD |
| IMA10 | | max | | DDGGIDIAPAA |
| G. max | 68 | Glycine | | MALTSKAINQECKKHACGNKDGDWYLYYAPTACTEGDDH |
| IMA16 | | max | | KGNRDSCFGHIAYMKGDDDGDIYDYAPAA |
| G. max | 69 | Glycine | | MALTSKAINQECKKHACGNKDGDWYLYYAPTACTEGDDH |
| IMA12 | | max | | KGNGDSCFGHIAYMKGDDDSDIYDYAPAA |
| G. max | 70 | Glycine | | MAFISMAINLIDCMTHACGNKDNDWYLYAPTACTEGDDP |
| IMA7 | | max | | MGDVDSWFAYME [symbol] GY [symbol] Y [symbol] |
| G. max | 71 | Glycine | | MASMSKAMTPEIKKHACDKKDGVSYHYDPTACAEGDDYN |
| IMA9 | | max | | GNINYVFAYME [symbol] DGGY [symbol] Y [symbol] |
| G. max | 72 | Glycine | | MASMSKAMTPEIKKHACDKKDGVLYHYDPTACAEGDDYN |
| IMA13 | | max | | GNINYVFAYME [symbol] DGGY [symbol] Y [symbol] |
| G. max | 73 | Glycine | | MTPEIKKHACDKKDGVLYHYDPTACAEGDDYNGNINYVF |
| IMA8 | | max | | AYME [symbol] DGGY [symbol] Y [symbol] |
| G. max | 74 | Glycine | | MSFTSKVIALWCKKHGNDDGVDVYDAPAATACIEGNVCN |
| IMA5 | | max | | WHGDFVSFVPVALVE [symbol] DDGGY [symbol] Y [symbol] |
| | | | | (C-motif: SEQ ID NO: 7) |
| G. max | 75 | Glycine | | MSFTSKVIAPWCKKHGNDDVVDAPAATTFIGGNVCNWHG |
| IMA4 | | max | | DFVSFVPIAYME [symbol] DGGY [symbol] Y [symbol] |
| | | | | (C-motif: SEQ ID NO: 6) |
| G. hirsutum | 76 | Gossypium | | MSPFSKVVASSCKKHVDGDYDDNDGFDYAPIACMEGNGD |
| IMA1 | | hirsutum | | DDDDDDYDYAPAASLD [symbol] DSY [symbol] Y [symbol] |
| J. curcas | 77 | Jatropha | | MSSVLLKAIASSWCNNQNLIIYDDGFDYASVVPSIDGDG |
| IMA1 | | curcas | | GDDDDGDYDYAPAASME [symbol] DDDDG [symbol] |
| J. curcas | 78 | Jatropha | | MVIVDSKKLGFFRLVAGEGQAMACFCMSKQND [symbol] |
| IMA2 | | curcas | | DDDDGGA [symbol] V [symbol] |
| L. japonicus | 79 | Lotus | | MVVLVCKESRLPKFFMAPPELQSFVIQNESDSDDDDDGD |
| IMA1 | | japonicus | | NDIDIAPAA |
| M. truncatula | 80 | Medicago | MTR7g | MSSISNVVAPWCKKHGNDHDGCVVWYDYPPTVCDE [symbol] |
| IMA15 | | truncatula | 087600 | [symbol] DGGY [symbol] Y [symbol] |
| M. truncatula | 81 | Medicago | MTR2g | MVFISMVIALNCKQHAYGEGEVDWFGCTSVSCIEEDYHN |
| IMA1 | | truncatula | 084210 | TDHDSYWE [symbol] DGGY [symbol] Y [symbol] |
| M. truncatula | 82 | Medicago | MTR2g | MASISMVIALNCKQHAYGEGNWFDYTSVSCIEEDYHNGD |
| IMA2 | | truncatula | 084215 | RDSYQE [symbol] DGGY [symbol] Y [symbol] |
| M. truncatula | 83 | Medicago | MTR2g | MASIFTVIAPLCKQNACGEGNGDWFGYTSVSCIVEDYRN |
| IMA3 | | truncatula | 084180 | GDQDSYKE [symbol] DGGY [symbol] Y [symbol] |
| M. truncatula | 84 | Medicago | | MTFISTVIAPKCKQYAYNGEGDGDWFGYTCVSCIEEDYT |
| IMA4 | | truncatula | | NGDRNLYRE [symbol] DGGY [symbol] Y [symbol] |
| M. truncatula | 85 | Medicago | MTR2g | MASISLAIIPKCEQHGYGEGNGDWISYTCVSCIEENYHN |
| IMA5 | | truncatula | 084170 | GDRDSCKE [symbol] DGGY [symbol] Y [symbol] |
| M. truncatula | 86 | Medicago | MTR2g | MASISLAITPKCKHHGYSEGNGDWFGYTSVSCIKEDNRN |
| IMA6 | | truncatula | 084190 | GDRDSCKEGDDDDDGGYDYAPTA |
| M. truncatula | 87 | Medicago | MTR2g | MASISMAITPKCKEHGYDEGNGDWFGYTYVSCIEEDYRN |
| IMA7 | | truncatula | 084200 | GDRDSYME [symbol] DGGY [symbol] Y [symbol] |
| M. truncatula | 88 | Medicago | MTR2g | MASISMVIAPKCKQHAFNGEGDCDWFGYTNISCIEEDYY |
| IMA8 | | truncatula | 084140 | NGE [symbol] DDGGY [symbol] Y [symbol] |
| M. truncatula | 89 | Medicago | MTR4g | MASISIAIATKSIKNVYDGGEWFGYASVACIEDYHIGDV |
| IMA9 | | truncatula | 026430 | DSYKE [symbol] DGGY [symbol] Y [symbol] |
| M. truncatula | 90 | Medicago | MTR4g | MASISIAIATRSIKHVYDEDEWFGYAASVACIEDYHTED |
| IMA10 | | truncatula | 026390 | VDSSYKE [symbol] DGGY [symbol] Y [symbol] |
| M. truncatula | 91 | Medicago | MTR4g | MASISIPTRSIKNACGEGEWFGFASVSCNDEDYHTGDVD |
| IMA11 | | truncatula | 026440 | SYRE [symbol] DGDY [symbol] Y [symbol] |
| M. truncatula | 92 | Medicago | MTR4g | MAFISISIATRSFQNACDEGEWFCYASVGCIEQDNHIGE |
| IMA12 | | truncatula | 026380 | EDSYRE [symbol] DGGY [symbol] Y [symbol] |
| M. truncatula | 93 | Medicago | | MTSISMVNISPKCNHAAYGECDGDWFGYASTICIKGNYY |
| IMA13 | | truncatula | | FRNEDSGSAHLIAYIE [symbol] DGGY [symbol] Y [symbol] |
| M. truncatula | 94 | Medicago | MTR2g | MASISIVNTAPKCNHAAYGECDVDSFGYASTVCIKGNYY |
| IMA14 | | truncatula | 084195 | IRNEDSGSADLVTYME [symbol] DGGY [symbol] Y [symbol] |
| M. notabilis | 95 | Morus | | MPHITFMNMVTARNGKDGDNGDHCYDHYFQYYNLSAPGE |
| IMA1 | | notabilis | | GDGDDDNDDDDDSGYDYAPAA |
| M. notabilis | 96 | Morus | | MFPAVDKLIYSESLSKKREDGGNHEDGNDGISRRYAPTQ |
| IMA2 | | notabilis | | VMENIYGASDRDYNYLPNATMD [symbol] DDDDSSY [symbol] Y |
| | | | | [symbol] |
| M. notabilis | 97 | Morus | | MSPVSEKNVAILAIMLCKKQNITYGDVTNGDFEYNLDPV |
| IMA3 | | notabilis | | TRIE [symbol] DDDDD [symbol] Y [symbol] |
| O. sativa | 98 | Oryza | LOC_O | MAPVSEASPLVHQDGGIIASFAVYAGAPCCSARGRMAET |
| IMA1 | | sativa | s01g459 | D [symbol] DDY [symbol] |
| | | | 14 | (C-motif: SEQ ID NO: 8) |
| O. sativa | 99 | Oryza | LOC_O | MAIAKSECERLAWALLLESNLLVGNRRSN [symbol] DDV |
| IMA2 | | sativa | s07g049 | [symbol] V [symbol] |
| | | | 20 | (C-motif: SEQ ID NO: 9) |
| P. sativum | 100 | Pisum | | MVIITSMESTLPMFLMDNHLSATEFICFCNQSKSE [symbol] |
| IMA1 | | sativum | | [symbol] GDI [symbol] I [symbol] |
| P. vulgaris | 101 | Phaseolus | PHAVU | MAFTSKLIAPCCNNHHALHQNHHAPPTFIEESVFNGHGD |
| IMA1 | | vulgaris | 003G16 | SVSFVPAASME [symbol] DDGSY [symbol] Y [symbol] |
| | | | 0300 | |
| P. vulgaris | 102 | Phaseolus | PHAVU | MLLFISCTKSGLPLFIKGNDWPETIFPLHHE [symbol] D |
| IMA2 | | vulgaris | 006G02 | DDDGGI [symbol] V [symbol] |
| | | | 8300 | |
| P. vulgaris | 103 | Phaseolus | PHAVU | MMFFVCKEYGLVLSNDWPATHDFHNFHE [symbol] DNGV |
| IMA3 | | vulgaris | 008G19 | I [symbol] V [symbol] |
| | | | 6700 | |
| P. trichocarpa | 104 | Populus | | MSPLSKTIIAFCTIHRHADGDDDGEYGYDYAPAACMEGD |
| IMA1 | | trichocarpa | | GDDDDSDYDYAPAAPME [symbol] DGDY [symbol] Y [symbol] |
| R. communis | 105 | Ricinus | | MSLFAISKAITISCCNKLADDCDNGDGCYFAPPPCIEGD |
| IMA2 | | communis | | GDDDDGDYDYAPAASSE [symbol] DDDGY [symbol] |
| R. communis | 106 | Ricinus | | MVHMAFLTARSSGKCSSTNLADQNKDIDVLFGYNEFSME |
| IMA1 | | communis | | APLIEDDGDGDDDDDDGGYDFAPAATLEGD [symbol] DG |
| | | | | DY [symbol] Y [symbol] |
| R. communis | 107 | Ricinus | | MSPLSKVIASWCNKPVVEEEMGNDDDRVYEYAPATSTE |
| IMA3 | | communis | | [symbol] DDDDDK [symbol] |
| S. lycopersicum | 108 | Solanum | | MTEIYSFHLYNKEILTRPVISFCLNRELENDDDDDDDGK |
| IMA1 | | lycopersicum | | KVAPAA |
| S. lycopersicum | 109 | Solanum | Solyc07 | MVIVRGNTTPFLPGRIEARPVINFGLNREFEADDDDDDD |
| IMA2 | | lycopersicum | g044900 | DDDGKKVAPAA |
| | | | 1 | |
| S. lycopersicum | 110 | Solanum | Solyc07 | MVIVRGNTSRFHPYEIEARLVISFYLNRELENDVDDDDD |
| IMA3 | | lycopersicum | g044910 | DDDDGAKVAPAA |
| | | | 1 | |
| S. lycopersicum | 111 | Solanum | Solyc12 | MSQISTILMNSICNFNHDVGSHVYESRSMMDRHATGCIY |
| IMA4 | | lycopersicum | g006720 | VSATWFEDD [symbol] DDDDADY [symbol] Y [symbol] |
| | | | 1 | |
| S. lycopersicum | 112 | Solanum | Solyc12 | MSTIFMIIGFEKRRSCADGDYDYTSAASLEGDDDDGDYG |
| IMA5 | | lycopersicum | g006730 | YAPAASLEGNDDDGDYDYAPAASLE [symbol] GDY [symbol] |
| | | | 1 | [symbol] |
| S. lycopersicum | 113 | Solanum | Solyc12 | MFGIFKIIGFEKIRRSCLDGDDDGDYDYAPAACLKRNGD |
| IMA6 | | lycopersicum | g006750 | DDGDYDYAPAAFLEGDDDDRDYDCAPAATIDGDDDGDYD |
| | | | 1 | YAPAA |
| S. lycopersicum | 114 | Solanum | Solyc12 | MSGILKIIGFEKIRRSCLDGDDDSDYDYAPAACLERDGD |
| IMA7 | | lycopersicum | g006760 | DDGDYDYAPAASLEGDDDDRDYDYVPAASLEGDDDGDYD |
| | | | 1 | YAPSGCMK |
| S. lycopersicum | 115 | Solanum | Solyc12 | MSGIFKIIGFEKIKRSCLDGDDDGDYDFAPAACLERDGD |
| IMA8 | | lycopersicum | g006770 | DDGDYDYAPAASLEGDDDDRDYDYVPAASLEGDDDGDYD |
| | | | 1 | YAPAA |
| S. lycopersicum | 116 | Solanum | Solyc12 | MSSIFKIIGFQKRRSCSDGDDDGDYDYAPSACLEGGGDG |
| IMA9 | | lycopersicum | g006780 | DDGDYDYTPAASLEGDCNDQDYDYAPAVSFEGHDVDGDY |
| | | | 1 | DYAPAA |
| S. tuberosum | 117 | Solanum | | MAGKSGRKVVRGVSKSSKAVYFWKIRHGCGIIFGKSKKY |
| IMA1 | | tuberosum | | KSCSYGSEDDDDDEHDYAPATYLERDDDDDDGNYDYAPA |
| | | | | ALT |
| S. tuberosum | 118 | Solanum | PGSC00 | MVVIMNANNKKVSLGCPDKFMATEAKLGSTICIDKECEA |
| IMA3 | | tuberosum | 03DMG | DDDYNDDDASKIAPAA |
| | | | 4000130 | |
| | | | 51 | |
| S. tuberosum | 119 | Solanum | | MSGTFKIIGFQKRRSSSDGDDEGYNYAPVTCLEGDGDDN |
| IMA4 | | tuberosum | | DAYYDYASAPFLEGDDDDGDYDYAPATSLEGEDNDGDYD |
| | | | | YAPAA |
| S. tuberosum | 120 | Solanum | | MSTIFKIIGFQKRRSCSDGDDDSDDGYDYAPAACLEGDG |
| IMA5 | | tuberosum | | DDNDGDYDYAPAASLEGDDDDGDYDYAPAASLE [symbol] |
| | | | | [symbol] GDY [symbol] Y [symbol] |
| S. tuberosum | 121 | Solanum | | MSTIFKIIGFEKRRSCPDGNYNYTLVASLEGDDDDGDYD |
| IMA6 | | tuberosum | | YAPAASLEGDDDDGDYDYAPAASLE [symbol] GNY [symbol] Y [symbol] |
| | | | | [symbol] |
| S. tuberosum | 122 | Solanum | | MSSIFKIIGFQNKRSYSDGDDDGDYDFAPAAFLEGDDDD |
| IMA7 | | tuberosum | | GDYDYAPAASLNGDDDDGDYDYVPAASLD [symbol] GDY |
| | | | | [symbol] Y [symbol] |
| S. tuberosum | 123 | Solanum | | MSSIFKIIGFHNRRSYSDGDDDGDYDYAPAAYLEGDDDD |
| IMA8 | | tuberosum | | EDYDYAPAASLNGDDDDGDYDYAPAASLE [symbol] GDY |
| | | | | [symbol] Y [symbol] |
| S. tuberosum | 124 | Solanum | | MSSIFKIIGFQNRRSYSDGDDDGDYDYAPTAYLEGDDDD |
| IMA9 | | tuberosum | | GDYDYAPVASLNGDDDDGDYDYAPATSLE [symbol] GDY |
| | | | | [symbol] Y [symbol] |
| S. tuberosum | 125 | Solanum | | MSSIFKIIGFEKRRSCLDGDDDGDYDYAPAACLERDGDD |
| IMA10 | | tuberosum | | DGDYDYAPAASLEGDDDDRDYDYAPAASLEGDDDGDYDY |
| | | | | APAA |
| S. tuberosum | 126 | Solanum | | MSSIFKIIGFQKRRLCLDGDDDGDYDYAPAACLEGGGDR |
| IMA11 | | tuberosum | | DDGDYYYALAASLEGDDDDRDYDYAPAASLGGDGDDGDY |
| | | | | DYAPVA |
| S. tuberosum | 127 | Solanum | | MSQISTILMNSICNLTFFDYHVERGNHDIGSHVYESTSM |
| IMA12 | | tuberosum | | MDRHVIGCIYVSATWFEDD [symbol] DDDDDDADY [symbol] Y [symbol] |
| | | | | [symbol] |
| S. tuberosum | 128 | Solanum | | MSEIFTIIGFEKIRRSCLDGDDDGDYDYAPASCLERDGD |
| IMA13 | | tuberosum | | DDGDYDYAPAASLEGDDDDRDYDYVPAASLEGDDDGDYD |
| | | | | YAPAA |
| S. tuberosum | 129 | Solanum | | MSSGIFKILGFEKRRLCSYGDDDGDYVYAPAACLNRDGD |
| IMA14 | | tuberosum | | DDGEYDYAPAASLEGDDDDRDYDYAPATNLEGDDDRDYD |
| | | | | YASAA |
| T. cacao | 130 | Theobroma | | MSSSKCIMHDEDNIKKIGSSSKNIMNDDVDHHKGRRDGY |
| IMA2 | | cacao | | VSNSKSLVQGGNSYTHVPSASVDGD [symbol] DDDY [symbol] F |
| | | | | [symbol] |
| T. urartu | 131 | Triticum | TRIUR3 | MAPASKVMSHVVQDGGIADYAVYAAAPCDAWCGGRHRKA |
| IMA1 | | urartu | 01690 | ESD [symbol] DDY [symbol] |
| T. urartu | 132 | Triticum | TRIUR3 | MAPASKIMSHIVVQDGGIAAYAVYAAAPCDAWCGGRHRK |
| IMA2 | | urartu | 18332 | AESD [symbol] DDY [symbol] |
| T. urartu | 133 | Triticum | TRIUR3 | MAPASKAMSHIVQDGGIATYAVYAAPCDAWCGGRHRKAE |
| IMA3 | | urartu | 29839 | TD [symbol] DDY [symbol] |
| V. vinifera | 134 | Vitis | | MSSISMAIDSQSMMHDGHVRGEHDKHGHVYCTNDNGGCY |
| IMA1 | | vinifera | | YALTAPREGD [symbol] DGDGGY [symbol] |
| Z. mays | 135 | Zea mays | | MAPVSSEAASYLVLIKGGSIAASSRAVYPWDGCSARGRM |
| IMA1 | | | | TETDSDDDDDDYDCAPAA |

2.3 Characterization of AtIMA1 Expression Pattern, Subcellular Localization, and Effects of AtIMA1 Overexpression on Fe Homeostasis Genes.

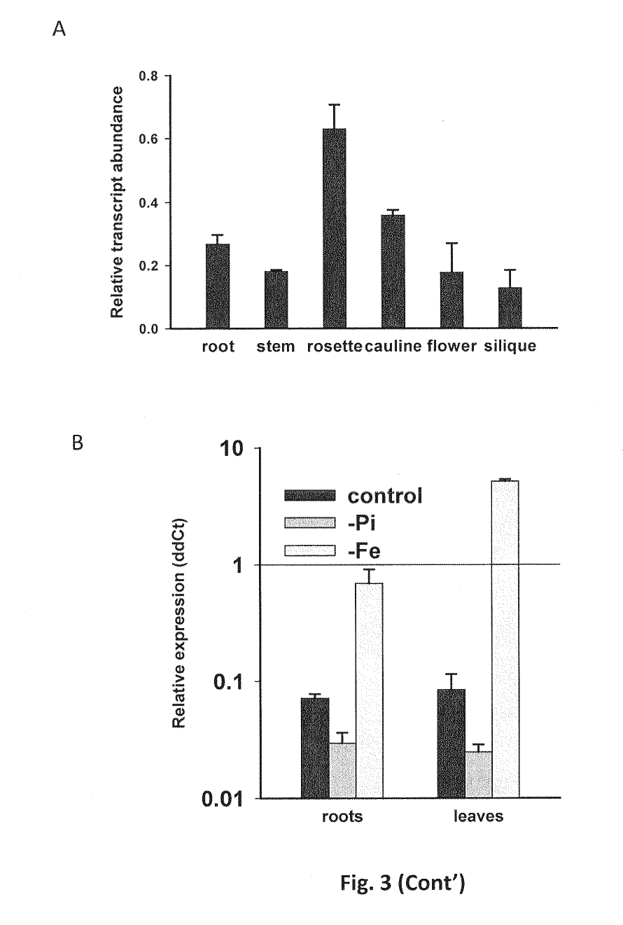

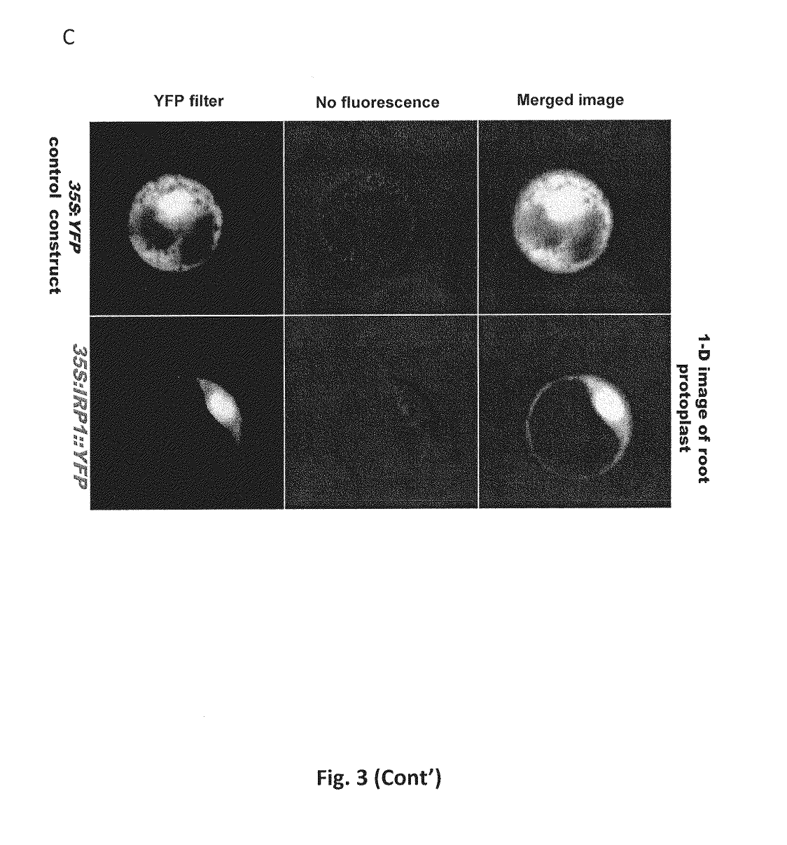

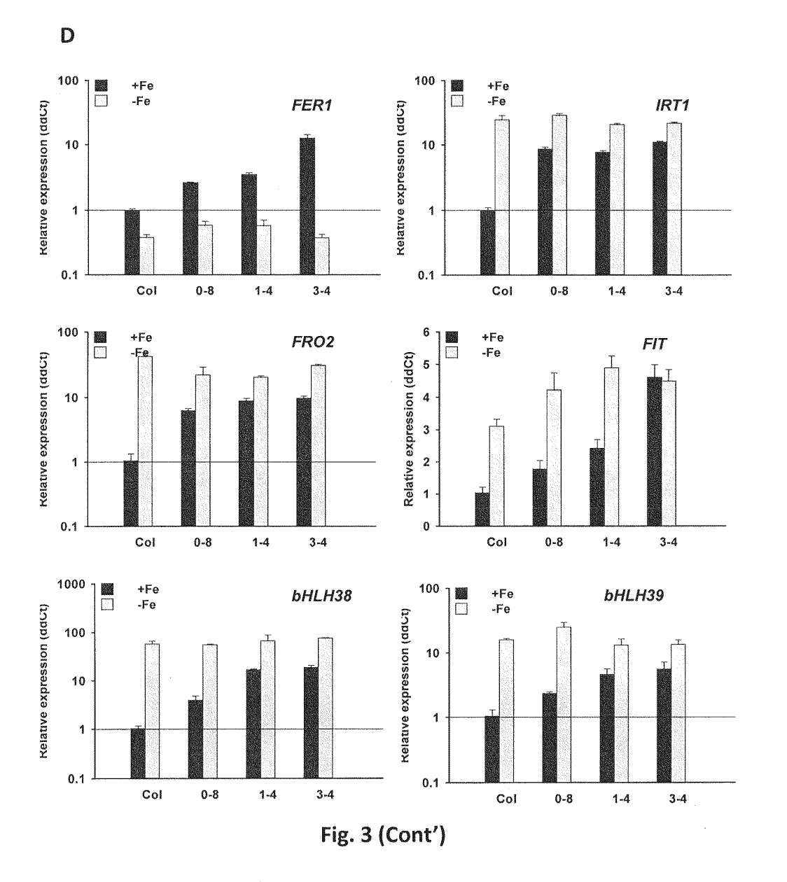

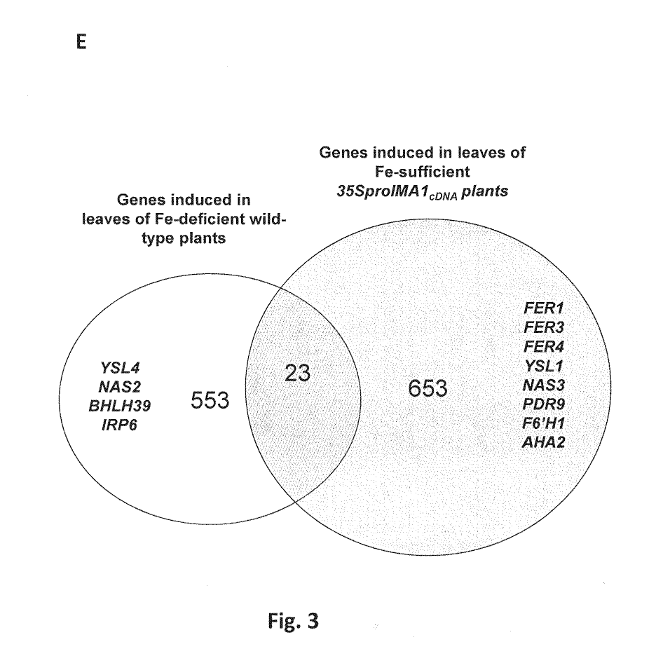

Expression analysis of AtIMA1 revealed ubiquitous gene activity throughout the plant with highest transcript levels in leaves (FIG. 3A). Growing the plants on Fe-deplete media for three days increased AtIMA1 transcripts approximately 10-fold in roots and 60-fold in leaves (FIG. 3B). Phosphate starvation, by contrast, which increases Fe levels (21), decreased AtIMA1 transcript levels (FIG. 3B), indicating that the expression of AtIMA1 is strictly dependent on the plant's Fe status and that induction of the gene is specific for Fe. AtIMA1 does not possess any targeting signal peptide and is predicted to localize to the cytoplasm and the nucleus. Recombinant IMA1:YFP expressed in Arabidopsis protoplasts showed strong signals in nuclei and in the cytoplasm, where it could either bind a receptor, recruit transcription factors, or act as an Fe chaperone (FIG. 3C).

In roots of 35Spro::AtIMA1_{cDNA} plants, the Fe acquisition genes AtlRT1 and AtFRO2 were strongly up-regulated under Fe-replete conditions (FIG. 3D). Importantly, the level of mRNA of the transcriptional regulators AtbHLH38, AtbHLH39 and AtFIT was also constitutively elevated when compared with the wild type (FIG. 3D). For example, the level of AtFIT transcript was 1.8- to 4.6-fold increased in three independent transgenic lines relative to the wild type. IMA thus appear to act upstream of the heterodimeric AtFIT/AtbHLH38/39 transcription regulators. Transcriptional profiling of leaves using the ATH1 microarray showed that genes that were previously shown to be important for Fe uptake by roots at high pH and low Fe solubility, namely the H⁺-ATPase AtAHA2 (22) and genes involved in the production and secretion of Fe-binding coumarins (At4CL2, AtF6′H1 and AtPDR9; 6-8), were constitutively up-regulated in leaves of 35Spro::AtIMA1_{cDNA} plants, indicative of a possible role of the encoded proteins not only in the uptake of Fe from the soil solution but also in the uptake of apoplasmic Fe by leaf cells (FIG. 3E). A role of coumarins in the protection of Fe overload caused by the up-regulated Fe uptake genes 35Spro::AtIMA1_{cDNA} plants is a plausible alternative scenario.

2.4 Effect of Heterologous Expression of AtIMA1 in Tomato Plants.

To explore whether IMA function is conserved, we generated transgenic tomato plants carrying the Arabidopsis 35Spro::AtIMA1_{cDNA} construct. MicroTom tomato plants ectopically expressing AtIMA1 grew normally without symptoms of Fe overload. Analysis of the fruit Fe concentration revealed a 60% increase in Fe levels (FIG. 4A), indicating that AtIMA1 is functional in tomato and that IMA is an integral and ubiquitous component of Fe signaling pathway in plants.

### REFERENCES

1. T. Kobayashi, N. K. Nishizawa, Annu. Rev. Plant Biol. 63, 131-152 (2012).
2. N. J. Robinson, C. M. Procter, E. L. Connolly, M. L. Guerinot, Nature 397, 694-697 (1999).
3. D. Eide, M. Broderius, J. Fett, M. L. Guerinot, Proc. Natl. Acad. Sci. USA 93, 5624-5628 (1996).
4. V. Römheld, H. Marschner, Plant Physiol 80, 175-180 (1986).
5. Y. Ishimaru, et al., Plant J. 45, 335-346 (2006).
6. J. Rodríguez-Celma, et al., Plant Physiol. 162, 1473-1485 (2013).
7. P. Fourcroy, et al., New Phytol. 201, 155-167 (2014).
8. N. B. Schmid, et al., Plant Physiol. 164, 160-172 (2014).
9. R. Ivanov, T. Brumbarova, P. Bauer, Mol. Plant 5, 27-42 (2012).
10. Y. Yuan, Cell Res. 18, 385-397 (2008).
11. N. Wang, Mol. Plant 6, 503-513 (2013).
12. Y. Ogo et al., Plant J. 51, 366-377 (2007).
13. T. Kobayashi, et al., S. Nat. Commun. 4, 2792 (2013).
14. D. Selote, R. Samira, A. Matthiadis, J. W. Gillikin, T. A. Long, Plant Physiol 167, 273-286 (2015).
15. L. Zheng, Plant Physiol. 151, 262-274 (2009).
16. J. Rodríguez-Celma et al., Front. Plant Sci. 4, 276 (2013).
17. L. Grillet, et al., J. Biol. Chem. 289, 2515-2525 (2014).
18. T. J. Buckhout, T. J. Yang, W. Schmidt, BMC Genomics 10, 147 (2009).
19. A. Zamboni, BMC Genomics 13, 101 (2012).
20. A. N. Moran Lauter et al., BMC Genomics 15, 702 (2014).
21. J. Misson, et al., Proc. Natl. Acad. Sci. USA 102, 11934-11939 (2005).
22. S. Santi, W. Schmidt, New Phytol. 183, 1072-1084 (2009).
23. W. D. Lin, et al., Plant Physiol. 155, 1383-1402 (2011).
24. T. L. Bailey et al., Nucleic Acids Res. 37, W202-W208 (2009).
25. P. Lan, W. Li, W. Schmidt, Mol. Cell. Proteomics 11, 1156-1166 (2012).
26. M. Karimi, D. Inze, A. Depicker, Trends Plant Sci. 7, 193-195 (2002).
27. R. Schwab, S. Ossowski, M. Riester, N. Warthmann, D. Weigel, Plant Cell 18, 1121-1133 (2006).
28. S. J. Clough, A. F. Bent, Plant J. 16, 735-743 (1998).
29. H. Roschzttardtz, G. Conejero, C. Curie, S. Mari, Plant Physiol. 151, 1329-1338 (2009).

1. A transgenic plant transformed with a recombinant polynucleotide comprising a nucleotide sequence encoding an iron-regulated polypeptide, operatively linked to an expression control sequence,
wherein the iron-regulated polypeptide comprises a C-terminal motif comprising from N-terminal to C-terminal
a first domain of GDDDD (SEQ ID NO: 1), and
a second domain of DXAPAA (SEQ ID NO: 2),
in which the first domain and said second domain are joined by a peptide spacer of 10 or less amino acid residues,
wherein the iron-regulated polypeptide comprises a total of 20 to 100 amino acid residues in length.

2. The transgenic plant of claim 1, wherein the iron-regulated polypeptide activates one or more transcriptional factors for Fe homeostasis in plants, selected from the group consisting of AtbHLH38, AtbHLH39, AtFIT and any combinations thereof.

3. The transgenic plant of claim 1, wherein the transgenic plant overexpresses the iron-regulated polypeptide and has a content of a trace element higher than that present in a control plant, where the trace element is selected from the group consisting of iron (Fe), zinc (Zn) and manganese (Mn).

4. The transgenic plant of claim 1, wherein the iron-regulated polypeptide comprises a total of 20 to 90, 25 to 85 or 45 to 75 amino acid residues in length.

5. The transgenic plant of claim 4, wherein the peptide space has a total of 1 to 6 or 1 to 3 amino acid residues.

6. The transgenic plant of claim 4, wherein the C-terminal motif comprises the amino acid sequence selected from the group consisting of:
(SEQ ID NO: 3)
GDDDDSGYDYAPAA;
(SEQ ID NO: 4)
GDDDDDDCDVAPAA;
(SEQ ID NO: 5)
GDDDDDDNGVIDVAPAA;
(SEQ ID NO: 6)
GDDDDDGGYDYAPAA;
(SEQ ID NO: 7)
GDDDDDDGGYDYAPAA;
(SEQ ID NO: 8)
GDDDDDDYDCAPAA;
and
(SEQ ID NO: 9)
GDDDDDDVDVAPAA.

7. The transgenic plant of claim 1, wherein the iron-regulated polypeptide comprises the amino acid sequence selected from the group consisting of: SEQ ID NOs: 25, 26, 27, 63, 65, 74, 75, 98 and 99.

8. The transgenic plant of claim 1, wherein the transgenic plant is monocotyledon or dicotyledon.

9. The transgenic plant of claim 1, wherein the transgenic plant comprises a plant part selected from the group consisting of leaves, shoots, roots, fruits and seeds.

10. The transgenic plant of claim 1, wherein the plant part is edible.

11. A plant tissue, plant part or plant cell of a transgenic plant of claim 1.

12. A method for biofortification comprising growing a transgenic plant of claim 1 or its seed or other propagating materials under a condition to express the iron-regulated polypeptide, sufficient for a content of a trace element to increase in the transgenic plant, wherein the trace element is selected from the group consisting of iron (Fe), zinc (Zn) and manganese (Mn).

13. The method of claim 12, wherein the iron-regulated polypeptide activates one or more transcriptional factors for Fe homeostasis in plants, selected from the group consisting of AtbHLH38, AtbHLH39, AtFIT and any combinations thereof.

14. The method of claim 12, wherein the iron-regulated polypeptide comprises a total of 20 to 90, 25 to 85 or 45 to 75 amino acid residues in length.

15. The method of claim 14, wherein the peptide space has a total of 1 to 6 or 1 to 3 amino acid residues.

16. The method of claim 12, wherein the C-terminal motif comprises the amino acid sequence selected from the group consisting of:
(SEQ ID NO: 3)
GDDDDSGYDYAPAA;
(SEQ ID NO: 4)
GDDDDDDNGVIDVAPAA;
(SEQ ID NO: 5)
GDDDDDGGYDYAPAA;
(SEQ ID NO: 6)
GDDDDDDGGYDYAPAA;
(SEQ ID NO: 7)
GDDDDDDYDCAPAA;
and
(SEQ ID NO: 8)
GDDDDDDVDVAPAA.

17. The method of claim 12, wherein the iron-regulated polypeptide comprises the amino acid sequence selected from the group consisting of: SEQ ID NOs: 25, 26, 27, 63, 65, 74, 75, 98 and 99.

18. A plant product made from a transgenic plant of claim 1 or a composition comprising the plant product.

19. The composition or plant product of claim 18, wherein the composition is a nutritional supplement or a pharmaceutical composition.

20. A method of supplementing a trace element in a subject, comprising administering an effective amount of a transgenic plant of claim 1 or a plant product or a composition of claim 18.
